SEQUENCE LISTING (22 sequences)

SEQ ID NO: 1 - DNA, 1697 bp, respiratory syncytial virus
atggagttgctaatcctcaaagcaaatgcaattaccacaatcctcactgcagtcacatttgttttgcttctggtcaaaacatcactgaagaattttatcaatcaacatgcagtgcagtagcaaaggctatcttagtgctctgagaactggttggtataccagtgttataactatagattaagtaatatcaaggaaaataagtgtaatggaacagatgctaaggtaaaattgataaacaagaattagataaatataaaaatgctgtaacagaattgcagttgctcatgcaaagcacccagcaacaaacaatcgagccagaagagaactaccaaggtttatgaattatacactcaaaatgccaaaaaaaccaatgtaacattaagcaagaaaaggaaaagaagatttcttggtttttgttaggtgttggatctgcaatcgccagtggcgttgctgtatctaaggtcctgcacctgaaggggaagtgaacaagatcaaaagtgctctactatccacaaacaaggctgtagtcagttatcaaatggagttagtgtcttaaccagcaaagtgttagacctcaaaaactatatagaaaacaattgttacctattgtgaacaagcaaagctgcagcatatcaaatatagcaactgtatagagttccaacaaaagaacaacagactactagagattaccagggaatttagtgttaagcaggtgtaactacacctgtaagcacttacatgttaactaatagtgaattattgtcattatcaatgatatgcctataacaaatgatcagaaaaagttaatgtccaacaatgttcaaatgttagacagcaaagttactctatcatgtccataataaaagaggaagtcttagcatatgtgtacaattaccactatatggtgttatagatacaccctgttggaaactacacacatccccctatgtacaaccaacacaaaagaagggtccaacatctgtttaacaagaactgacagaggtggtactgtgacaatgcaggatcagtatctttcttcccacaagctgaaacatgtaaagtcaatcaaatcgagtattttgtgacacaatgaacagtttaacattaccaagtgaagtaaactctgcaatgttgacatattcaaccccaaatatgattgtaaaattatgacttcaaaaacgatgtaagcagctccgttatcacatctctaggagccattgtgtcatgctatggcaaaacaaatgtacagcatccaataaaaatcgtggaatcataaagacattttctaacgggtgcgatatgtatcaaataaaggggtggacactgtgtctgtaggtaacacattatattatgtaaaaagcaagaaggtaaaagtctctatgtaaaaggtgaaccaataataaatttctatgacccttagtattcccctctgatgaatttgatgcatcaatatctcaagtcaacgagaagattaacagagcctagcatttattcgtaaatccgatgaattattacataatgtaaatgctggtaatccaccataaatatcatgataactactataattatagtgattatagtaatattgttatcttaattgctgttggactgctcttatactgtaaggccagaagcacaccagtcacactaagaaagatcaactgagtggtataaataatattgcatttagtaactaa

SEQ ID NO: 2 - protein, 574 aa, respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTINIMITTIIIVIIVILLSLIAVGLLLYCKARSTPVTLSKDQLSGINNIAFSN

SEQ ID NO: 3 - DNA, 897 bp, respiratory syncytial virus
atgtccaaaaacaaggaccaacgcaccgctaagacactagaaaagacctgggacactctcaatcatttattattcatatcatcgggcttatataagttaaatcttaaatctatagcacaaatcacattatccattctggcaatgataatctcaacttcacttataattacagccatcatattcatagcctcggcaaaccacaaagtcacactaacaactgcaatcatacaagatgcaacaagccagatcaagaacacaaccccaacatacctcactcaggatcctcagcttggaatcagcttctccaatctgtctgaaattacatcacaaaccaccaccatactagcttcaacaacaccaggagtcaagtcaaacctgcaacccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatatgcagcaacaatccaacctgctgggctatctgcaaaagaataccaaacaaaaaaccaggaaagaaaaccaccaccaagcctacaaaaaaaccaaccttcaagacaaccaaaaaagatctcaaacctcaaaccactaaaccaaaggaagtacccaccaccaagcccacagaagagccaaccatcaacaccaccaaaacaaacatcacaactacactgctcaccaacaacaccacaggaaatccaaaactcacaagtcaaatggaaaccttccactcaacctcctccgaaggcaatctaagcccttctcaagtctccacaacatccgagcacccatcacaaccctcatctccacccaacacaacacgccagtag

SEQ ID NO: 4 - protein, 298 aa, respiratory syncytial virus
MSKNKDQRTAKTLEKTWDTLNHLLFISSGLYKLNLKSIAQITLSILAMIISTSLIITAIIFIASANHKVTLTTAIIQDATSQIKNTTPTYLTQDPQLGISFSNLSEITSQTTTILASTTPGVKSNLQPTTVKTKNTTTTQTQPSKPTTKQRQNKPPNKPNNDFHFEVFNFVPCSICSNNPTCWAICKRIPNKKPGKKTTTKPTKKPTFKTTKKDLKPQTTKPKEVPTTKPTEEPTINTTKTNITTTLLTNNTTGNPKLTSQMETFHSTSSEGNLSPSQVSTTSEHPSQPSSPPNTTRQ

SEQ ID NO: 5 - DNA, 1566 bp, Artificial Sequence
  Recombinant PreF polynucleotide
aagcttgccaccatggagctgctgatcctgaaaaccaacgccatcaccgccatcctggccgccgtgaccctgtgcttcgcctcctcccagaacatcaccgaggagttctaccagtccacctgctccgccgtgtccaagggctacctgtccgccctgcggaccggctggtacacctccgtgatcaccatcgagctgtccaacatcaaggaaaacaagtgcaacggcaccgacgccaaggtgaagctgatcaagcaggagctggacaagtacaagagcgccgtgaccgaactccagctgctgatgcagtccacccctgccaccaacaacaagtttctgggcttcctgctgggcgtgggctccgccatcgcctccggcatcgccgtgagcaaggtgctgcacctggagggcgaggtgaacaagatcaagagcgccctgctgtccaccaacaaggccgtggtgtccctgtccaacggcgtgtccgtgctgacctccaaggtgctggatctgaagaactacatcgacaagcagctgctgcctatcgtgaacaagcagtcctgctccatctccaacatcgagaccgtgatcgagttccagcagaagaacaaccggctgctggagatcacccgcgagttctccgtgaacgccggcgtgaccacccctgtgtccacctacatgctgaccaactccgagctgctgtccctgatcaacgacatgcctatcaccaacgaccagaaaaaactgatgtccaacaacgtgcagatcgtgcggcagcagtcctacagcatcatgagcatcatcaaggaagaggtgctggcctacgtggtgcagctgcctctgtacggcgtgatcgacaccccttgctggaagctgcacacctcccccctgtgcaccaccaacaccaaggagggctccaacatctgcctgacccggaccgaccggggctggtactgcgacaacgccggctccgtgtccttcttccctctggccgagacctgcaaggtgcagtccaaccgggtgttctgcgacaccatgaactccctgaccctgccttccgaggtgaacctgtgcaacatcgacatcttcaaccccaagtacgactgcaagatcatgaccagcaagaccgacgtgtcctccagcgtgatcacctccctgggcgccatcgtgtcctgctacggcaagaccaagtgcaccgcctccaacaagaaccggggaatcatcaagaccttctccaacggctgcgactacgtgtccaataagggcgtggacaccgtgtccgtgggcaacacactgtactacgtgaataagcaggagggcaagagcctgtacgtgaagggcgagcctatcatcaacttctacgaccctctggtgttcccttccgacgagttcgacgcctccatcagccaggtgaacgagaagatcaaccagtccctggccttcatccggaagtccgacgagaagctgcataacgtggaggacaagatcgaggagatcctgtccaaaatctaccacatcgagaacgagatcgcccggatcaagaagctgatcggcgaggcctgataatctaga

SEQ ID NO: 6 - protein, 514 aa, Artificial Sequence
  Recombinant PreF Antigen
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEKLHNVEDKIEEILSKIYHIENEIARIKKLIGEA

SEQ ID NO: 7 - DNA, 1855 bp, Artificial Sequence
  Chimeric PreF-G Antigen polynucleotide seqeunce
aagcttgccaccatggagctgctgatcctcaagaccaacgccatcaccgccatcctggccgccgtgaccctgtgcttcgcctcctcccagaacatcaccgaagagttctaccagtccacctgctccgccgtgtccaagggctacctgtccgccctgcggaccggctggtacacctccgtgatcaccatcgagctgtccaacatcaaagaaaacaagtgcaacggcaccgacgccaaggtcaagctgatcaagcaggaactggacaagtacaagagcgccgtgaccgaactccagctgctgatgcagtccacccctgccaccaacaacaagaagtttctgggcttcctgctgggcgtgggctccgccatcgcctccggcatcgccgtgagcaaggtgctgcacctggagggcgaggtgaacaagatcaagagcgccctgctgtccaccaacaaggccgtggtgtccctgtccaacggcgtgtccgtgctgacctccaaggtgctggatctgaagaactacatcgacaagcagctgctgcctatcgtgaacaagcagtcctgctccatctccaacatcgagaccgtgatcgagttccagcagaagaacaaccggctgctggagatcacccgcgagttctccgtgaacgccggcgtgaccacccctgtgtccacctacatgctgacaaactccgagctgctctccctgatcaacgacatgcctatcaccaacgaccaaaaaaagctgatgtccaacaacgtgcagatcgtgcggcagcagtcctacagcatcatgagcatcatcaaggaagaagtcctggcctacgtcgtgcagctgcctctgtacggcgtgatcgacaccccttgctggaagctgcacacctcccccctgtgcaccaccaacaccaaagagggctccaacatctgcctgacccggaccgaccggggctggtactgcgacaacgccggctccgtgtccttcttccctctggccgagacctgcaaggtgcagtccaaccgggtgttctgcgacaccatgaactccctgaccctgccttccgaggtgaacctgtgcaacatcgacatcttcaaccccaagtacgactgcaagatcatgaccagcaagaccgacgtgtcctccagcgtgatcacctccctgggcgccatcgtgtcctgctacggcaagaccaagtgcaccgcctccaacaagaaccggggaatcatcaagaccttctccaacggctgcgactacgtgtccaataagggcgtggacaccgtgtccgtgggcaacacactgtactacgtgaataagcaggaaggcaagagcctgtacgtgaagggcgagcctatcatcaacttctacgaccctctggtgttcccttccgacgagttcgacgcctccatcagccaggtcaacgagaagatcaaccagtccctggccttcatccggaagtccgacgagaagctgcataacgtggaggacaagatcgaagagatcctgtccaaaatctaccacatcgagaacgagatcgcccggatcaagaagctgatcggcgaggctggcggctctggcggcagcggcggctccaagcagcggcagaacaagcctcctaacaagcccaacaacgacttccacttcgaggtgttcaacttcgtgccttgctccatctgctccaacaaccctacctgctgggccatctgcaagagaatccccaacaagaagcctggcaagaaaaccaccaccaagcctaccaagaagcctaccttcaagaccaccaagaaggaccacaagcctcagaccacaaagcctaaggaagtgccaaccaccaagcaccaccaccatcaccactgataatcta

SEQ ID NO: 8 - protein, 605 aa, Artificial Sequence
  Chimeric PreF-G polypeptide
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKKFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEKLHNVEDKIEEILSKIYHIENEIARIKKLIGEAGGSGGSGGSKQRQNKPPNKPNNDFHFEVFNFVPCSICSNNPTCWAICKRIPNKKPGKKTTTKPTKKPTFKTTKKDHKPQTTKPKEVPTTK

SEQ ID NO: 9 - DNA, 1834 bp, Artificial Sequence
  Chimeric PreF-G polynucleotide
aagcttgccaccatggagctgctgatcctcaagaccaacgccatcaccgccatcctggccgccgtgaccctgtgcttcgcctcctcccagaacatcaccgaagagttctaccagtccacctgctccgccgtgtccaagggctacctgtccgccctgcggaccggctggtacacctccgtgatcaccatcgagctgtccaacatcaaagaaaacaagtgcaacggcaccgacgccaaggtcaagctgatcaagcaggaactggacaagtacaagagcgccgtgaccgaactccagctgctgatgcagtccacccctgccaccaacaacaagaagtttctgggcttcctgctgggcgtgggctccgccatcgcctccggcatcgccgtgagcaaggtgctgcacctggagggcgaggtgaacaagatcaagagcgccctgctgtccaccaacaaggccgtggtgtccctgtccaacggcgtgtccgtgctgacctccaaggtgctggatctgaagaactacatcgacaagcagctgctgcctatcgtgaacaagcagtcctgctccatctccaacatcgagaccgtgatcgagttccagcagaagaacaaccggctgctggagatcacccgcgagttctccgtgaacgccggcgtgaccacccctgtgtccacctacatgctgacaaactccgagctgctctccctgatcaacgacatgcctatcaccaacgaccaaaaaaagctgatgtccaacaacgtgcagatcgtgcggcagcagtcctacagcatcatgagcatcatcaaggaagaagtcctggcctacgtcgtgcagctgcctctgtacggcgtgatcgacaccccttgctggaagctgcacacctcccccctgtgcaccaccaacaccaaagagggctccaacatctgcctgacccggaccgaccggggctggtactgcgacaacgccggctccgtgtccttcttccctctggccgagacctgcaaggtgcagtccaaccgggtgttctgcgacaccatgaactccctgaccctgccttccgaggtgaacctgtgcaacatcgacatcttcaaccccaagtacgactgcaagatcatgaccagcaagaccgacgtgtcctccagcgtgatcacctccctgggcgccatcgtgtcctgctacggcaagaccaagtgcaccgcctccaacaagaaccggggaatcatcaagaccttctccaacggctgcgactacgtgtccaataagggcgtggacaccgtgtccgtgggcaacacactgtactacgtgaataagcaggaaggcaagagcctgtacgtgaagggcgagcctatcatcaacttctacgaccctctggtgttcccttccgacgagttcgacgcctccatcagccaggtcaacgagaagatcaaccagtccctggccttcatccggaagtccgacgagaagctgcataacgtggaggacaagatcgaagagatcctgtccaaaatctaccacatcgagaacgagatcgcccggatcaagaagctgatcggcgaggctggcggcaagcagcggcagaacaagcctcctaacaagcccaacaacgacttccacttcgaggtgttcaacttcgtgccttgctccatctgctccaacaaccctacctgctgggccatctgcaagagaatccccaacaagaagcctggcaagaaaaccaccaccaagcctaccaagaagcctaccttcaagaccaccaagaaggaccacaagcctcagaccacaaagcctaaggaagtgccaaccaccaagcaccaccaccatcaccactgataatcta

SEQ ID NO: 10 - protein, 598 aa, Artificial Sequence
  Chimeric PreF-G polypeptide
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKKFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEKLHNVEDKIEEILSKIYHIENEIARIKKLIGEAGGKQRQNKPPNKPNNDFHFEVFNFVPCSICSNNPTCWAICKRIPNKKPGKKTTTKPTKKPTFKTTKKDHKPQTTKPKEVPTTK

SEQ ID NO: 11 - protein, 28 aa, Artificial Sequence
  Isoleucine substituted GCN4 leucine zipper
EDKIEEILSKIYHIENEIARIKKLIGEA

SEQ ID NO: 12 - DNA, 1563 bp, Artificial sequence
  Codon optimized PreF nucleotide sequence
atggagctgcccatcctgaagaccaacgccatcaccaccatcctcgccgccgtgaccctgtgcttcgccagcagccagaacatcacggaggagttctaccagagcacgtgcagcgccgtgagcaagggctacctgagcgcgctgcgcacgggctggtacacgagcgtgatcacgatcgagctgagcaacatcaaggagaacaagtgcaacggcacggacgcgaaggtgaagctgatcaagcaggagctggacaagtacaagagcgcggtgacggagctgcagctgctgatgcagagcacgccggcgacgaacaacaagttcctcggcttcctgctgggcgtgggcagcgcgatcgcgagcggcatcgccgtgagcaaggtgctgcacctggagggcgaggtgaacaagatcaagtccgcgctgctgagcacgaacaaggcggtcgtgagcctgagcaacggcgtgagcgtgctgacgagcaaggtgctcgacctgaagaactacatcgacaagcagctgctgccgatcgtgaacaagcagagctgcagcatcagcaacatcgagaccgtgatcgagttccagcagaagaacaaccgcctgctggagatcacgcgggagttctccgtgaacgcaggcgtgacgacgcccgtgtctacgtacatgctgacgaacagcgagctgctcagcctgatcaacgacatgccgatcacgaacgaccagaagaagctgatgagcaacaacgtgcagatcgtgcgccagcagagctacagcatcatgagcatcatcaaggaggaggtgctggcatacgtggtgcagctgccgctgtacggcgtcatcgacacgccctgctggaagctgcacacgagcccgctgtgcacgaccaacacgaaggagggcagcaacatctgcctgacgcggacggaccggggctggtactgcgacaacgcgggcagcgtgagcttcttcccgctcgcggagacgtgcaaggtgcagagcaaccgcgtcttctgcgacacgatgaacagcctgacgctgccgagcgaggtgaacctgtgcaacatcgacatcttcaacccgaagtacgactgcaagatcatgacgagcaagaccgatgtcagcagcagcgtgatcacgagcctcggcgcgatcgtgagctgctacggcaagacgaagtgcacggcgagcaacaagaaccgcggcatcatcaagacgttcagcaacggctgcgactatgtgagcaacaagggcgtggacactgtgagcgtcggcaacacgctgtactacgtgaacaagcaggagggcaagagcctgtacgtgaagggcgagccgatcatcaacttctacgacccgctcgtgttcccgagcgacgagttcgacgcgagcatcagccaagtgaacgagaagatcaaccagagcctggcgttcatccgcaagagcgacgagaagctgcacaacgtggaggacaagatcgaggagatcctgagcaagatctaccacatcgagaacgagatcgcgcgcatcaagaagctgatcggcgaggcgcatcatcaccatcaccattga

SEQ ID NO: 13 - DNA, 1685 bp, Artificial sequence
  PreF polynucleotide sequence with intron
atggagctgctgatcctgaaaaccaacgccatcaccgccatcctggccgccgtgaccctgtgcttcgcctcctcccagaacatcaccgaggagttctaccagtccacctgctccgccgtgtccaagggctacctgtccgccctgcggaccggctggtacacctccgtgatcaccatcgagctgtccaacatcaaggaaaacaagtgcaacggcaccgacgccaaggtgaagctgatcaagcaggagctggacaagtacaagagcgccgtgaccgaactccagctgctgatgcagtccacccctgccaccaacaacaagtttctgggcttcctgctgggcgtgggctccgccatcgcctccggcatcgccgtgagcaaggtacgtgtcgggacttgtgttcccctttttttaataaaaagttatatctttaatgttatatacatatttcctgtatgtgatccatgtgcttatgactttgtttatcatgtgtttaggtgctgcacctggagggcgaggtgaacaagatcaagagcgccctgctgtccaccaacaaggccgtggtgtccctgtccaacggcgtgtccgtgctgacctccaaggtgctggatctgaagaactacatcgacaagcagctgctgcctatcgtgaacaagcagtcctgctccatctccaacatcgagaccgtgatcgagttccagcagaagaacaaccggctgctggagatcacccgcgagttctccgtgaacgccggcgtgaccacccctgtgtccacctacatgctgaccaactccgagctgctgtccctgatcaacgacatgcctatcaccaacgaccagaaaaaactgatgtccaacaacgtgcagatcgtgcggcagcagtcctacagcatcatgagcatcatcaaggaagaggtgctggcctacgtggtgcagctgcctctgtacggcgtgatcgacaccccttgctggaagctgcacacctcccccctgtgcaccaccaacaccaaggagggctccaacatctgcctgacccggaccgaccggggctggtactgcgacaacgccggctccgtgtccttcttccctctggccgagacctgcaaggtgcagtccaaccgggtgttctgcgacaccatgaactccctgaccctgccttccgaggtgaacctgtgcaacatcgacatcttcaaccccaagtacgactgcaagatcatgaccagcaagaccgacgtgtcctccagcgtgatcacctccctgggcgccatcgtgtcctgctacggcaagaccaagtgcaccgcctccaacaagaaccggggaatcatcaagaccttctccaacggctgcgactacgtgtccaataagggcgtggacaccgtgtccgtgggcaacacactgtactacgtgaataagcaggagggcaagagcctgtacgtgaagggcgagcctatcatcaacttctacgaccctctggtgttcccttccgacgagttcgacgcctccatcagccaggtgaacgagaagatcaaccagtccctggccttcatccggaagtccgacgagaagctgcataacgtggaggacaagatcgaggagatcctgtccaaaatctaccacatcgagaacgagatcgcccggatcaagaagctgatcggcgaggccggaggtcaccaccaccatcaccactga

SEQ ID NO: 14 - protein, 9 aa, Artificial sequence
  Synthetic linker peptide
GGSGGSGGS

SEQ ID NO: 15 - protein, 4 aa, Artificial sequence
  Furin cleavage consensus motif
RARR

SEQ ID NO: 16 - protein, 4 aa, Artificial sequence
  Furin cleavage consensus motif
RKRR

SEQ ID NO: 17 - DNA, 1542 bp, Artificial Sequence
  Synthetic PreF Polynucleotide
atggagctgctgatcctgaaaaccaacgccatcaccgccatcctggccgccgtgaccctgtgcttcgcctcctcccagaacatcaccgaggagttctaccagtccacctgctccgccgtgtccaagggctacctgtccgccctgcggaccggctggtacacctccgtgatcaccatcgagctgtccaacatcaaggaaaacaagtgcaacggcaccgacgccaaggtgaagctgatcaagcaggagctggacaagtacaagagcgccgtgaccgaactccagctgctgatgcagtccacccctgccaccaacaacaagtttctgggcttcctgctgggcgtgggctccgccatcgcctccggcatcgccgtgagcaaggtgctgcacctggagggcgaggtgaacaagatcaagagcgccctgctgtccaccaacaaggccgtggtgtccctgtccaacggcgtgtccgtgctgacctccaaggtgctggatctgaagaactacatcgacaagcagctgctgcctatcgtgaacaagcagtcctgctccatctccaacatcgagaccgtgatcgagttccagcagaagaacaaccggctgctggagatcacccgcgagttctccgtgaacgccggcgtgaccacccctgtgtccacctacatgctgaccaactccgagctgctgtccctgatcaacgacatgcctatcaccaacgaccagaaaaaactgatgtccaacaacgtgcagatcgtgcggcagcagtcctacagcatcatgagcatcatcaaggaagaggtgctggcctacgtggtgcagctgcctctgtacggcgtgatcgacaccccttgctggaagctgcacacctcccccctgtgcaccaccaacaccaaggagggctccaacatctgcctgacccggaccgaccggggctggtactgcgacaacgccggctccgtgtccttcttccctctggccgagacctgcaaggtgcagtccaaccgggtgttctgcgacaccatgaactccctgaccctgccttccgaggtgaacctgtgcaacatcgacatcttcaaccccaagtacgactgcaagatcatgaccagcaagaccgacgtgtcctccagcgtgatcacctccctgggcgccatcgtgtcctgctacggcaagaccaagtgcaccgcctccaacaagaaccggggaatcatcaagaccttctccaacggctgcgactacgtgtccaataagggcgtggacaccgtgtccgtgggcaacacactgtactacgtgaataagcaggagggcaagagcctgtacgtgaagggcgagcctatcatcaacttctacgaccctctggtgttcccttccgacgagttcgacgcctccatcagccaggtgaacgagaagatcaacgggaccctggccttcatccggaagtccgacgagaagctgcataacgtggaggacaagatcgaggagatcctgtccaaaatctaccacatcgagaacgagatcgcccggatcaagaagctgatcggcgaggcc

SEQ ID NO: 18 - protein, 514 aa, Artificial Sequence
  Synthetic PreF Polypeptide
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINGTLAFIRKSDEKLHNVEDKIEEILSKIYHIENEIARIKKLIGEA

SEQ ID NO: 19 - DNA, 1542 bp, Artificial Sequence
  Synthetic PreF Polynucleotide
atggagctgctgatcctgaaaaccaacgccatcaccgccatcctggccgccgtgaccctgtgcttcgcctcctcccagaacatcaccgaggagttctaccagtccacctgctccgccgtgtccaagggctacctgtccgccctgcggaccggctggtacacctccgtgatcaccatcgagctgtccaacatcaaggaaaacaagtgcaacggcaccgacgccaaggtgaagctgatcaagcaggagctggacaagtacaagagcgccgtgaccgaactccagctgctgatgcagtccacccctgccaccaacaacaagtttctgggcttcctgcagggcgtgggctccgccatcgcctccggcatcgccgtgagcaaggtgctgcacctggagggcgaggtgaacaagatcaagagcgccctgctgtccaccaacaaggccgtggtgtccctgtccaacggcgtgtccgtgctgacctccaaggtgctggatctgaagaactacatcgacaagcagctgctgcctatcgtgaacaagcagtcctgctccatctccaacatcgagaccgtgatcgagttccagcagaagaacaaccggctgctggagatcacccgcgagttctccgtgaacgccggcgtgaccacccctgtgtccacctacatgctgaccaactccgagctgctgtccctgatcaacgacatgcctatcaccaacgaccagaaaaaactgatgtccaacaacgtgcagatcgtgcggcagcagtcctacagcatcatgagcatcatcaaggaagaggtgctggcctacgtggtgcagctgcctctgtacggcgtgatcgacaccccttgctggaagctgcacacctcccccctgtgcaccaccaacaccaaggagggctccaacatctgcctgacccggaccgaccggggctggtactgcgacaacgccggctccgtgtccttcttccctctggccgagacctgcaaggtgcagtccaaccgggtgttctgcgacaccatgaactccctgaccctgccttccgaggtgaacctgtgcaacatcgacatcttcaaccccaagtacgactgcaagatcatgaccagcaagaccgacgtgtcctccagcgtgatcacctccctgggcgccatcgtgtcctgctacggcaagaccaagtgcaccgcctccaacaagaaccggggaatcatcaagaccttctccaacggctgcgactacgtgtccaataagggcgtggacaccgtgtccgtgggcaacacactgtactacgtgaataagcaggagggcaagagcctgtacgtgaagggcgagcctatcatcaacttctacgaccctctggtgttcccttccgacgagttcgacgcctccatcagccaggtgaacgagaagatcaaccagtccctggccttcatccggaagtccgacgagaagctgcataacgtggaggacaagatcgaggagatcctgtccaaaatctaccacatcgagaacgagatcgcccggatcaagaagctgatcggcgaggcc

SEQ ID NO: 20 - protein, 514 aa, Artificial Sequence
  Synthetic PreF Polypeptide
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKFLGFLQGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEKLHNVEDKIEEILSKIYHIENEIARIKKLIGEA

SEQ ID NO: 21 - DNA, 1542 bp, Artificial Sequence
  Synthetic PreF Polynucleotide
atggagctgctgatcctgaaaaccaacgccatcaccgccatcctggccgccgtgaccctgtgcttcgcctcctcccagaacatcaccgaggagttctaccagtccacctgctccgccgtgtccaagggctacctgtccgccctgcggaccggctggtacacctccgtgatcaccatcgagctgtccaacatcaaggaaaacaagtgcaacggcaccgacgccaaggtgaagctgatcaagcaggagctggacaagtacaagagcgccgtgaccgaactccagctgctgatgcagtccacccctgccaccaacaacaagtttctgggcttcctgcagggcgtgggctccgccatcgcctccggcatcgccgtgagcaaggtgctgcacctggagggcgaggtgaacaagatcaagagcgccctgctgtccaccaacaaggccgtggtgtccctgtccaacggcgtgtccgtgctgacctccaaggtgctggatctgaagaactacatcgacaagcagctgctgcctatcgtgaacaagcagtcctgctccatctccaacatcgagaccgtgatcgagttccagcagaagaacaaccggctgctggagatcacccgcgagttctccgtgaacgccggcgtgaccacccctgtgtccacctacatgctgaccaactccgagctgctgtccctgatcaacgacatgcctatcaccaacgaccagaaaaaactgatgtccaacaacgtgcagatcgtgcggcagcagtcctacagcatcatgagcatcatcaaggaagaggtgctggcctacgtggtgcagctgcctctgtacggcgtgatcgacaccccttgctggaagctgcacacctcccccctgtgcaccaccaacaccaaggagggctccaacatctgcctgacccggaccgaccggggctggtactgcgacaacgccggctccgtgtccttcttccctctggccgagacctgcaaggtgcagtccaaccgggtgttctgcgacaccatgaactccctgaccctgccttccgaggtgaacctgtgcaacatcgacatcttcaaccccaagtacgactgcaagatcatgaccagcaagaccgacgtgtcctccagcgtgatcacctccctgggcgccatcgtgtcctgctacggcaagaccaagtgcaccgcctccaacaagaaccggggaatcatcaagaccttctccaacggctgcgactacgtgtccaataagggcgtggacaccgtgtccgtgggcaacacactgtactacgtgaataagcaggagggcaagagcctgtacgtgaagggcgagcctatcatcaacttctacgaccctctggtgttcccttccgacgagttcgacgcctccatcagccaggtgaacgagaagatcaacgggaccctggccttcatccggaagtccgacgagaagctgcataacgtggaggacaagatcgaggagatcctgtccaaaatctaccacatcgagaacgagatcgcccggatcaagaagctgatcggcgaggcc

SEQ ID NO: 22 - protein, 514 aa, Artificial Sequence
  Synthetic PreF Polypeptide
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKFLGFLQGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINGTLAFIRKSDEKLHNVEDKIEEILSKIYHIENEIARIKKLIGEA

(19) United States
(12) Patent Application Publication
(10) Pub. No.: US 2014/0037680 A1
(43) Pub. Date: Feb. 6, 2014

(54) NOVEL METHOD

(71) Applicant: GLAXOSMITHKLINE BIOLOGICALS, S.A., Rixensart (BE)

(72) Inventors: Ann-Muriel STEFF, Laval (CA); Jean-Francios TOUSSAINT, Rixensart (BE)

(73) Assignee: GLAXOSMITHKLINE BIOLOGICALS, S.A., Rixensart (BE)

(21) Appl. No.: 13/835,829

(22) Filed: Mar. 15, 2013

Related U.S. Application Data
(60) Provisional application No. 61/679,928, filed on Aug. 6, 2012.

(51) Int. Cl. A61K 39/155; A61K 39/08; A61K 39/02; A61K 39/05; A61K 45/06; A61K 39/145

(52) U.S. Cl. CPC A61K 39/155; A61K 45/06; A61K 39/145; A61K 39/099; A61K 39/05; A61K 39/08
USPC 424/201.1; 424/211.1; 424/202.1

(57) ABSTRACT

This disclosure provides methods for protecting infants against disease caused by respiratory syncytial virus (RSV) through maternal immunization using recombinant respiratory syncytial virus (RSV) antigens to reduce the incidence or severity of RSV infection in young infants.

### CROSS-REFERENCE TO RELATED APPLICATIONS

This application claims benefit of the filing date of U.S. Provisional Application No. 61/679,928, filed 6 Aug. 2012, the disclosure of which is incorporated herein by reference.

### COPYRIGHT NOTIFICATION PURSUANT TO 37 C.F.R. §1.71(E)

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

This disclosure concerns the field of immunology. More particularly this disclosure relates to compositions and methods for eliciting an immune response specific for Respiratory Syncytial Virus (RSV).

Human Respiratory Syncytial Virus (RSV) is the most common worldwide cause of lower respiratory tract infections (LRTI) in infants less than 6 months of age and premature babies less than or equal to 35 weeks of gestation.

RSV is well documented as a cause of yearly winter epidemics of acute LRTI, including bronchiolitis and pneumonia. The incidence rate of RSV-associated LRTI in otherwise healthy children was calculated as 37 per 1000 child-year in the first two years of life (45 per 1000 child-year in infants less than 6 months old) and the risk of hospitalization as 6 per 1000 child-years (per 1000 child-years in the first six months of life). Incidence is higher in children with cardio-pulmonary disease and in those born prematurely, who constitute almost half of RSV-related hospital admissions in the USA. Children who experience a more severe LRTI caused by RSV later have an increased incidence of childhood asthma. These studies demonstrate widespread need for RSV vaccines.

Various approaches have been attempted in efforts to produce a safe and effective RSV vaccine that produces durable and protective immune responses in healthy and at risk populations. However, none of the approaches evaluated to date have been proven safe and effective as a vaccine for the purpose of preventing RSV infection and/or reducing or preventing RSV disease, including LRTIs in neonates and young infants.

### BRIEF SUMMARY

The present disclosure relates to methods for protecting infants against disease caused by respiratory syncytial virus (RSV) by administering to pregnant women an immunogenic composition comprising a recombinant antigen that is an RSV F protein analog. Active immunization of pregnant women with an immunogenic composition containing an F protein analog elicits maternal antibodies, which are transferred to the gestational infant via the placenta, thereby protecting the infant from RSV disease following birth. Accordingly, the present disclosure also relates to methods of immunising a pregnant woman to protect her infant from disease caused by RSV, and the use of a recombinant RSV F protein analog in immunizing a pregnant female to protect her infant from disease caused by RSV.

### BRIEF DESCRIPTION OF THE DRAWINGS

FIG. 1A is a schematic illustration highlighting structural features of the RSV F protein. FIG. 1B is a schematic illustration of exemplary RSV Prefusion F (PreF) antigens.

FIG. 2 shows the study design for a guinea pig experiment performed in Example 1.

FIG. 3 shows results from Example 1 following challenge of guinea pig progeny with RSV.

FIG. 4 shows the time-course of the neutralizing antibody response in the guinea pig model in Example 1.

### DETAILED DESCRIPTION

### Introduction

A particular challenge in the development of a safe and effective vaccine that protects infants against disease caused by Respiratory Syncytial Virus (RSV) is that the highest incidence is in very young infants. This in itself presents many challenges. Young infants, especially those born prematurely, can have an immature immune system. There is also a hypothetical risk of interference of maternal antibodies with vaccination in very young infants. In the past there has been a problem with enhancement of RSV disease with vaccination of young infants against RSV. The present disclosure concerns methods of protecting young infants, e.g., between birth and 6 months of age, from disease caused by RSV by actively immunizing pregnant women with an immunogenic composition containing an analog of the RSV F protein. The F protein analog favorably elicits antibodies, which are transferred to the gestational infant via the placenta resulting in passive immunological protection of the infant following birth and lasting through the critical period for infection and severe disease caused by RSV.

One aspect of this disclosure relates to a method for protecting an infant against infection or disease caused by respiratory syncytial virus (RSV), the method comprising administering to a pregnant female with a gestational infant an immunogenic composition comprising a recombinant RSV antigen comprising an F protein analog, wherein at least one subset of maternal antibodies elicited by the immunogenic composition are transferred to the gestational infant, thereby protecting the infant against infection or disease caused by RSV.

In an embodiment, the disclosure concerns a method of immunizing a pregnant female to protect her infant from disease caused by RSV by administering to the pregnant female a recombinant RSV antigen comprising an F protein analog that is capable of boosting a humoral immune response specific for RSV. Upon boosting, at least one subset of maternal antibodies specific for RSV elicited by the immunogenic composition are transferred to the gestational infant, thereby protecting the infant against infection or disease caused by RSV.

In another aspect, the disclosure relates to a recombinant RSV antigen comprising an F protein analog for use in immunizing a pregnant female to protect her infant from infection or disease caused by RSV, wherein administration of the immunogenic composition to the pregnant female elicits at least one subset of maternal antibodies that are transferred to the gestational infant. It is viewed as advantageous that the subset of maternal antibodies includes neutralizing antibodies. Similarly, it is viewed as advantageous that the subset of maternal antibodies includes antibodies of the IgG₁ subclass.

In a particular embodiment the method or use involves administration of an F protein analog which is a prefusion F or “PreF” antigen that includes at least one modification that stabilizes the prefusion conformation of the F protein. Alternatively, F analogs stabilized in the postfusion conformation (“PostF”), or that are labile with respect to conformation can be employed in the methods described herein. Generally the F protein analog, e.g., PreF, PostF, etc.) antigen lacks a transmembrane domain, and is soluble, i.e., not membrane bound (for example, to facilitate expression and purification of the F protein analog).

In exemplary embodiments, the method or use involves the administration of an F protein analog that comprises in an N-terminal to C-terminal direction: at least a portion or substantially all of an F₂ domain and an F₁ domain of an RSV F protein polypeptide, optionally with a heterologous trimerization domain. In an embodiment, there is no furin cleavage site between the F₂ domain and the F₁ domain. In certain exemplary embodiments, the F₂ domain comprises at least a portion of an RSV F protein polypeptide corresponding to amino acids 26-105 of the reference F protein precursor polypeptide (F₀) of SEQ ID NO:2 and/or the F₁ domain comprises at least a portion of an RSV F protein polypeptide corresponding to amino acids 137-516 of the reference F protein precursor polypeptide (F₀) of SEQ ID NO:2.

For example, in specific embodiments, the F protein analog is selected from the group of:
a) a polypeptide comprising a polypeptide selected from the group of SEQ ID NO:6, SEQ ID NO:8, SEQ ID NO:10, SEQ ID NO:18, SEQ ID NO:20 and SEQ ID NO:22;
b) a polypeptide encoded by a polynucleotide selected from the group of SEQ ID NO:5, SEQ ID NO:7, SEQ ID NO:9, SEQ ID NO:17, SEQ ID NO:19 and SEQ ID NO:21, or by a polynucleotide sequence that hybridizes under stringent conditions over substantially its entire length to a polynucleotide selected from the group of SEQ ID NO:5, SEQ ID NO:7, SEQ ID NO:9, SEQ ID NO:17, SEQ ID NO:19 and SEQ ID NO:21, which polypeptide comprises an amino acid sequence corresponding at least in part to a naturally occurring RSV strain;
c) a polypeptide with at least 95% sequence identity to a polypeptide selected from the group of SEQ ID NO:6, SEQ ID NO:8, SEQ ID NO:10, SEQ ID NO:18, SEQ ID NO:20 and SEQ ID NO:22, which polypeptide comprises an amino acid sequence that does not correspond to a naturally occurring RSV strain.

Optionally, the F protein analog further comprises a signal peptide. Optionally, the F protein analog can further comprise a “tag” or sequence to facilitate purification, e.g., a multi-histidine sequence.

In embodiments comprising a heterologous trimerization domain, such a domain can comprise a coiled-coil domain, such as an isoleucine zipper, or it can comprise an alternative trimerization domain, such as from the bacteriophage T4 fibritin (“foldon”), or influenza HA.

In certain exemplary embodiments, the F protein analog comprises at least one modification selected from:

(i) a modification that alters glycosylation.

(ii) a modification that eliminates at least one non-furin cleavage site;

(iii) a modification that deletes one or more amino acids of the pep27 domain; and

(iv) a modification that substitutes or adds a hydrophilic amino acid in a hydrophobic domain of the F protein extracellular domain.

In certain embodiments, the F protein analog comprises a multimer of polypeptides, for example, a trimer of polypeptides.

In certain embodiments, the F protein analog is formulated in an immunogenic composition comprising a pharmaceutically acceptable carrier or excipient, such as a buffer. Optionally, the immunogenic composition also includes an adjuvant, for example, and adjuvant that includes 3D-MPL, QS21 (e.g., in a detoxified form), an oil-in-water emulsion (e.g., with or without immunostimulatory molecules, such as α-tocopherol), mineral salts, such as aluminium salts, including alum, or combinations thereof. Preferably, if the immunogenic composition includes an adjuvant, the adjuvant enhances or increases a humoral immune characterized by the production of antibodies, particularly IgG antibodies of the IgG₁ subclass.

Typically, the F protein analog is administered during the third trimester of gestation, although a beneficial effect (especially pregnancies at increased risk of preterm delivery) can be obtained prior to the beginning of the third trimester. Thus, although the F protein analog can be administered at 26 weeks of gestation (measured from the start of the last menstrual period) or later, for example between 26 and 38 weeks of gestation, or between 28 and 34 weeks of gestation, the F protein analog can also be administered prior to 26 weeks of gestation.

Favorably, administration of the immunogenic composition to the pregnant female elicits an immune response, which when passively transferred to her gestational infant, protects the infant, e.g., from birth to at least about 6 months of age. Accordingly, the infant protected by the methods disclosed herein can be an infant of less than six months of age, such as less than two months of age, for example, less than one month of age, e.g., a neonate or newborn. Favorably, at least one subset of antibodies, for example, IgG antibodies, such as IgG₁ antibodies are transferred via the placenta. Optionally, to ensure continuity of the immune response beyond six months of age, the method can involve administering to the infant one or more compositions that primes or induces an active immune response against RSV in the infant. The composition administered to the infant can be the same or different from that administered to the pregnant female. For example, the composition can also be an F protein analog or it can include a nucleic acid that encodes one or more RSV antigens, such as a live attenuated virus, a viral vector (e.g., an adenoviral or MVA vector), or a viral replicon particle or other self-replicating nucleic acid.

The methods and uses disclosed herein can reduce the incidence or severity of infection or disease caused by RSV, for example by reducing the incidence or severity of RSV disease such as LRTI, or by reducing the incidence of severe RSV disease such as LRTI. Protecting the infant favorably includes protecting the infant from severe disease and hospitalization caused by RSV. As such, the methods and uses disclosed herein can reduce the incidence of severe disease caused by RSV by 50% or more, or 60% or more, or 70% or more, as measured by a 50% or more, or 60% or more, or 70% or more reduction in the rate of severe LRTI in a cohort of infants of vaccinated mothers compared to infants of unvaccinated mothers.

### Terms

In order to facilitate review of the various embodiments of this disclosure, the following explanations of terms are provided. Additional terms and explanations can be provided in the context of this disclosure.

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “˜”) 200 pg.

Although methods and materials similar or equivalent to those described herein can be used in the practice or testing of this disclosure, suitable methods and materials are described below. The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof. The abbreviation, “e.g.” is derived from the Latin exempli gratia, and is used herein to indicate a non-limiting example. Thus, the abbreviation “e.g.” is synonymous with the term “for example.”

The term “F protein” or “Fusion protein” or “F protein polypeptide” or “Fusion protein polypeptide” refers to a polypeptide or protein having all or part of an amino acid sequence of an RSV Fusion protein polypeptide. Numerous RSV Fusion proteins have been described and are known to those of skill in the art. WO2008114149 sets out exemplary F protein variants (for example, naturally occurring variants).

An “F protein analog” refers to an F protein which includes a modification that alters the structure or function of the F protein but which retains the immunological properties of the F protein such that an immune response generated against an F protein analog will recognize the native F protein. WO2010149745, incorporated herein in its entirety by reference, sets out exemplary F protein analogs. WO2011008974, incorporated herein in its entirety by reference, also sets out exemplary F protein analogs. F protein analogs include for example PreF antigens which include at least one modification that stabilizes the prefusion conformation of the F protein and which are generally soluble, i.e., not membrane bound. F protein analogs also include post-fusion F (postF) antigens which are in the post-fusion conformation of the RSV F protein, favorably stabilized in such conformation. F analogs further include F protein in an intermediate conformation, favorably stabilized in such conformation. Such alternatives are also generally soluble.

A “variant” when referring to a nucleic acid or a polypeptide (e.g., an RSV F or G protein nucleic acid or polypeptide, or an F analog nucleic acid or polypeptide) is a nucleic acid or a polypeptide that differs from a reference nucleic acid or polypeptide. Usually, the difference(s) between the variant and the reference nucleic acid or polypeptide constitute a proportionally small number of differences as compared to the referent.

A “domain” of a polypeptide or protein is a structurally defined element within the polypeptide or protein. For example, a “trimerization domain” is an amino acid sequence within a polypeptide that promotes assembly of the polypeptide into trimers. For example, a trimerization domain can promote assembly into trimers via associations with other trimerization domains (of additional polypeptides with the same or a different amino acid sequence). The term is also used to refer to a polynucleotide that encodes such a peptide or polypeptide.

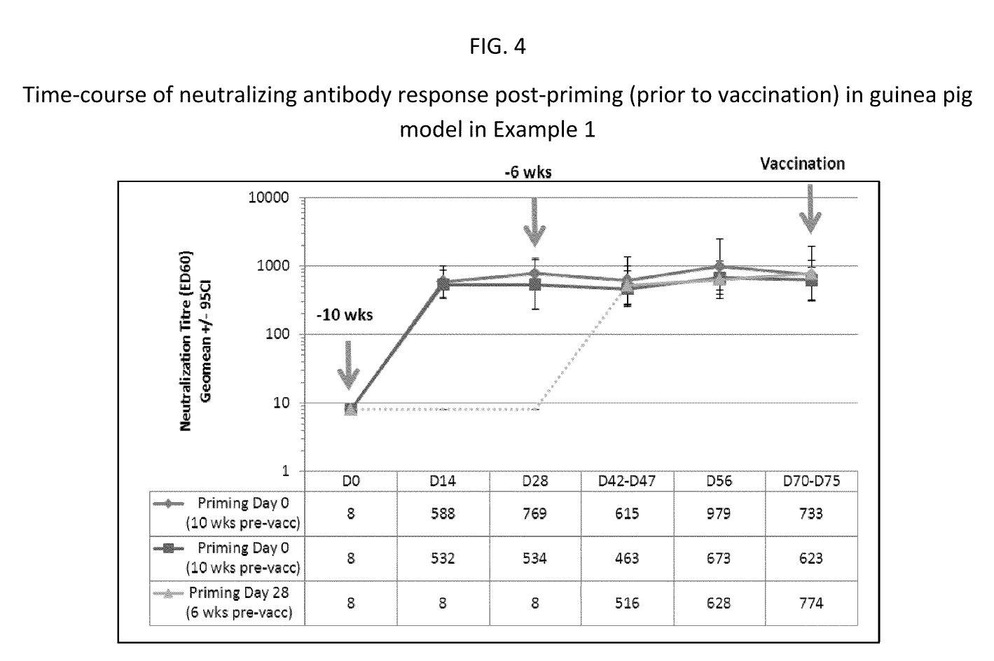

The terms “native” and “naturally occurring” refer to an element, such as a protein, polypeptide or nucleic acid that is present in the same state as it is in nature. That is, the element has not been modified artificially. It will be understood, that in the context of this disclosure, there are numerous native/naturally occurring variants of RSV proteins or polypeptides, e.g., obtained from different naturally occurring strains or isolates of RSV. WO2008114149, incorporated herein by reference in its entirety, contains exemplary RSV strains, proteins and polypeptides, see for example FIG. 4.

The term “polypeptide” refers to a polymer in which the monomers are amino acid residues which are joined together through amide bonds. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “fragment,” in reference to a polypeptide, refers to a portion (that is, a subsequence) of a polypeptide. The term “immunogenic fragment” refers to all fragments of a polypeptide that retain at least one predominant immunogenic epitope of the full-length reference protein or polypeptide. Orientation within a polypeptide is generally recited in an N-terminal to C-terminal direction, defined by the orientation of the amino and carboxy moieties of individual amino acids. Polypeptides are translated from the N or amino-terminus towards the C or carboxy-terminus.

A “signal peptide” is a short amino acid sequence (e.g., approximately 18-25 amino acids in length) that directs newly synthesized secretory or membrane proteins to and through membranes, e.g., of the endoplasmic reticulum. Signal peptides are frequently but not universally located at the N-terminus of a polypeptide, and are frequently cleaved off by signal peptidases after the protein has crossed the membrane. Signal sequences typically contain three common structural features: an N-terminal polar basic region (n-region), a hydrophobic core, and a hydrophilic c-region).

The terms “polynucleotide” and “nucleic acid sequence” refer to a polymeric form of nucleotides at least 10 bases in length. Nucleotides can be ribonucleotides, deoxyribonucleotides, or modified forms of either nucleotide. The term includes single and double forms of DNA. By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. In one embodiment, a polynucleotide encodes a polypeptide. The 5′ and 3′ direction of a nucleic acid is defined by reference to the connectivity of individual nucleotide units, and designated in accordance with the carbon positions of the deoxyribose (or ribose) sugar ring. The informational (coding) content of a polynucleotide sequence is read in a 5′ to 3′ direction.

A “recombinant” nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, e.g., by genetic engineering techniques. A “recombinant” protein is one that is encoded by a heterologous (e.g., recombinant) nucleic acid, which has been introduced into a host cell, such as a bacterial or eukaryotic cell. The nucleic acid can be introduced, on an expression vector having signals capable of expressing the protein encoded by the introduced nucleic acid or the nucleic acid can be integrated into the host cell chromosome.

The term “heterologous” with respect to a nucleic acid, a polypeptide or another cellular component, indicates that the component occurs where it is not normally found in nature and/or that it originates from a different source or species.

An “antigen” is a compound, composition, or substance that can stimulate the production of antibodies and/or a T cell response in a subject, including compositions that are injected, absorbed or otherwise introduced into a subject. The term “antigen” includes all related antigenic epitopes. The term “epitope” or “antigenic determinant” refers to a site on an antigen to which B and/or T cells respond. The “dominant antigenic epitopes” or “dominant epitope” are those epitopes to which a functionally significant host immune response, e.g., an antibody response or a T-cell response, is made. Thus, with respect to a protective immune response against a pathogen, the dominant antigenic epitopes are those antigenic moieties that when recognized by the host immune system result in protection from disease caused by the pathogen. The term “T-cell epitope” refers to an epitope that when bound to an appropriate MHC molecule is specifically bound by a T cell (via a T cell receptor). A “B-cell epitope” is an epitope that is specifically bound by an antibody (or B cell receptor molecule).

An “adjuvant” is an agent that enhances the production of an immune response in a non-antigen specific manner. Common adjuvants include suspensions of minerals (alum, aluminum hydroxide, aluminum phosphate) onto which antigen is adsorbed; emulsions, including water-in-oil, and oil-in-water (and variants thereof, including double emulsions and reversible emulsions), liposaccharides, lipopolysaccharides, immunostimulatory nucleic acids (such as CpG oligonucleotides), liposomes, Toll-like Receptor agonists (particularly, TLR2, TLR4, TLR7/8 and TLR9 agonists), and various combinations of such components.

An “antibody” or “immunoglobulin” is a plasma protein, made up of four polypeptides that binds specifically to an antigen. An antibody molecule is made up of two heavy chain polypeptides and two light chain polypeptides (or multiples thereof) held together by disulfide bonds. In humans, antibodies are defined into five isotypes or classes: IgG, IgM, IgA, IgD, and IgE. IgG antibodies can be further divided into four sublclasses (IgG₁, IgG₂, IgG₃ and IgG₄). A “neutralizing” antibody is an antibody that is capable of inhibiting the infectivity of a virus. Accordingly, a neutralizing antibodies specific for RSV are capable of inhibiting or reducing the infectivity of RSV.

An “immunogenic composition” is a composition of matter suitable for administration to a human or animal subject (e.g., in an experimental or clinical setting) that is capable of eliciting a specific immune response, e.g., against a pathogen, such as RSV. As such, an immunogenic composition includes one or more antigens (for example, polypeptide antigens) or antigenic epitopes. An immunogenic composition can also include one or more additional components capable of eliciting or enhancing an immune response, such as an excipient, carrier, and/or adjuvant. In certain instances, immunogenic compositions are administered to elicit an immune response that protects the subject against symptoms or conditions induced by a pathogen. In some cases, symptoms or disease caused by a pathogen is prevented (or reduced or ameliorated) by inhibiting replication of the pathogen (e.g., RSV) following exposure of the subject to the pathogen. In the context of this disclosure, the term immunogenic composition will be understood to encompass compositions that are intended for administration to a subject or population of subjects for the purpose of eliciting a protective or palliative immune response against RSV (that is, vaccine compositions or vaccines).

An “immune response” is a response of a cell of the immune system, such as a B cell, T cell, or monocyte, to a stimulus, such as a pathogen or antigen (e.g., formulated as an immunogenic composition or vaccine). An immune response can be a B cell response, which results in the production of specific antibodies, such as antigen specific neutralizing antibodies. An immune response can also be a T cell response, such as a CD4+ response or a CD8+ response. B cell and T cell responses are aspects of a “cellular” immune response. An immune response can also be a “humoral” immune response, which is mediated by antibodies. In some cases, the response is specific for a particular antigen (that is, an “antigen-specific response”). If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by the inhibition of viral replication or plaque formation in a plaque reduction assay or ELISA-neutralization assay, or by measuring resistance to pathogen challenge in vivo. Exposure of a subject to an immunogenic stimulus, such as a pathogen or antigen (e.g., formulated as an immunogenic composition or vaccine), elicits a primary immune response specific for the stimulus, that is, the exposure “primes” the immune response. A subsequent exposure, e.g., by immunization, to the stimulus can increase or “boost” the magnitude (or duration, or both) of the specific immune response. Thus, “boosting” a preexisting immune response by administering an immunogenic composition increases the magnitude of an antigen (or pathogen) specific response, (e.g., by increasing antibody titre and/or affinity, by increasing the frequency of antigen specific B or T cells, by inducing maturation effector function, or any combination thereof).

The adjective “pharmaceutically acceptable” indicates that the referent is suitable for administration to a subject (e.g., a human or animal subject). Remington's Pharmaceutical Sciences, by E. W. Martin, Mack Publishing Co., Easton, Pa., 15th Edition (1975), describes compositions and formulations (including diluents) suitable for pharmaceutical delivery of therapeutic and/or prophylactic compositions, including immunogenic compositions.

The term “reduces” is a relative term, such that an agent reduces a response or condition if the response or condition is quantitatively diminished following administration of the agent, or if it is diminished following administration of the agent, as compared to a reference agent. Similarly, the term “protects” does not necessarily mean that an agent completely eliminates the risk of an infection or disease caused by infection, so long as at least one characteristic of the response or condition is substantially or significantly reduced or eliminated. Thus, an immunogenic composition that protects against or reduces an infection or a disease, or symptom thereof, can, but does not necessarily prevent or eliminate infection or disease in all subjects, so long as the incidence or severity of infection or incidence or severity of disease is measurably reduced, for example, by at least about 50%, or by at least about 60%, or by at least about 70%, or by at least about 80%, or by at least about 90% of the infection or response in the absence of the agent, or in comparison to a reference agent. In certain instances, the reduction is in the incidence of lower respiratory tract infections (LRTI), or the incidence of severe LRTI, or hospitalizations due to RSV disease, or in the severity of disease caused by RSV.

A “subject” is a living multi-cellular vertebrate organism. In the context of this disclosure, the subject can be an experimental subject, such as a non-human animal, e.g., a mouse, a cotton rat, guinea pig, cow, or a non-human primate. Alternatively, the subject can be a human subject.

### Maternal Immunization

Lower respiratory tract infection (LRTI) is an infection of any of the tissues of the lower respiratory tract with the most severe forms including bronchiolitis and pneumonia. Human Respiratory Syncytial Virus (RSV) is the most common worldwide cause of lower respiratory tract infections (LRTI) in infants less than 6 months of age and premature babies less than or equal to 35 weeks of gestation. The RSV disease spectrum includes a wide array of respiratory symptoms from rhinitis and otitis to pneumonia and bronchiolitis, the latter two diseases being associated with considerable morbidity and mortality.

A particular challenge in the development of a safe and effective vaccine that protects infants against disease caused by Respiratory Syncytial Virus (RSV) is that the highest incidence is in very young infants. Young infants, especially those born prematurely, can have an immature immune system. Physiologically infants may be more susceptible to diseases of the lower respiratory tract than older children. Thus, protecting young infants from RSV disease and in particular from severe LRTI, is particularly important. There is also a hypothetical risk of interference of antibodies transferred via the placenta to the infant (“maternal antibodies”) with vaccination of the infant, such that vaccination in early infancy may not be sufficiently effective, e.g., to elicit a fully protective neutralizing antibody response.

The present disclosure concerns methods of protecting young infants from disease caused by RSV by actively immunizing pregnant women with a safe and effective immunogenic composition containing an analog of the RSV F protein. The F protein analog favorably elicits antibodies (e.g., neutralizing antibodies), by boosting or increasing the magnitude of the humoral response previously primed by natural exposure to (or prior vaccination against) RSV. The antibodies produced in response to the F protein analog are transferred to the gestational infant via the placenta, resulting in passive immunological protection of the infant following birth and lasting through the critical period for infection and severe disease caused by RSV. Typically, the passive immunological protection conferred by this method lasts between birth and at least two months of age, and preferably up to about 6 months of age, or even longer.

The immunogenic compositions described herein containing an F protein analog are designed to induce a strong neutralizing antibody response. Since pregnant mothers have typically been exposed to RSV one or more times during their lives, they have an existing primed response to RSV. The proportion of the population exposed to RSV infection by adulthood is essentially 100%. Therefore, the provision of protection from RSV for the infant immediately after birth and for the crucial first few months that follow, is achieved by boosting this primed response as effectively as possible to increase neutralizing antibody responses against RSV in the mother, and favorably in respect of particular antibody subclasses (subtypes) such as IgG₁ that can cross the placenta and provide protection to the infant. In one embodiment, immunogenic compositions for use herein do not include an adjuvant, or include an adjuvant which favors a strong IgG₁ response such as a mineral salt such as an aluminium or calcium salt, in particular aluminium hydroxide, aluminium phosphate or calcium phosphate. Thus in a particular embodiment the F protein analog for the methods and uses described herein is favorably formulated with a mineral salt, favorably alum. In alternative embodiments, the adjuvant that favors a strong IgG₁ response is an oil-in-water emulsion, or a saponin, such as QS21 (or a detoxified version thereof), as will be described in more detail below.

A pregnant female can be a human female, and accordingly, the infant or gestational infant can be a human infant. For a pregnant human female the gestational age of the developing fetus is measured from the start of the last menstrual period. The number of weeks post-conception is measured from 14 days after the start of the last menstrual period. Thus, when a pregnant human female is said to be 24 weeks post conception this will be equal to 26 weeks after the start of her last menstrual period, or 26 weeks of gestation. When a pregnancy has been achieved by assisted reproductive technology, gestational stage of the developing fetus is calculated from two weeks before the date of conception.

The term “gestational infant” as used herein means the fetus or developing fetus of a pregnant female. The term “gestational age” is used to mean the number of weeks of gestation i.e. the number of weeks since the start of the last menstrual period. Human gestation is typically about 40 weeks from the start of the last menstrual period, and may conveniently be divided into trimesters, with the first trimester extending from the first day of the last menstrual period through the 13^{th} week of gestation; the second trimester spanning from the 14^{th} through the 27^{th} weeks of gestation, and the third trimester starting in the 28^{th} week and extending until birth. Thus, the third trimester starts at 26 weeks post-conception and continues through to birth of the infant.

The term “infant” when referring to a human is between 0 and two years of age It will be understood that the protection provided by the methods and uses described herein can potentially provide protection for an infant into childhood, from aged 2 to 11, or early childhood for example from ages 2 to 5, or even into adolescence, from aged 12 to 18. However it is during infancy that an individual is most vulnerable to severe RSV disease so this is the focus of the present specification (e.g., from birth to about 6 months of age).

A human infant can be immunologically immature in the first few months of life, especially when born prematurely, e.g., before 35 weeks gestation, when the immune system may not be sufficiently well developed to mount an immune response capable of preventing infection or disease caused by a pathogen in the way that a developed immune system would be capable of doing in response to the same pathogen, An immunologically immature infant is more likely to succumb to infection and disease caused by a pathogen than an infant with a more developed or mature immune system. A human infant can also have an increased vulnerability to LRTIs during the first few months of life for physiological and developmental reasons, for example, airways are small and less developed or mature than in children and adults. For these reasons, when we refer herein to the first six months of infancy this may be extended for premature or pre-term infants according to the amount of time lost in gestational age below 40 weeks or below 38 weeks or below 35 weeks.

In an alternative embodiment, the pregnant female and its infant are from any species such as those described above under “subjects”. For a pregnant animal, such as a pregnant guinea pig, the time post-conception is measured as the time since mating. In humans and in some animals, for example guinea pigs, antibodies pass from the mother to the fetus via the placenta. Some antibody isotypes may be preferentially transferred through the placenta, for example in humans IgG₁ antibodies are the isotype most efficiently transferred across the placenta. Although subclasses exist in experimental animals, such as guinea pigs and mice, the various subclasses do not necessarily serve the same function, and a direct correlate between subclasses of humans and animals cannot easily be made.

Favorably, protecting the infant by reducing the incidence or severity of RSV infection covers at least the neonatal period and very young infancy, for example at least the first several weeks of life following birth, such as the first month from birth, or the first two months, or the first three months, or the first four months, or the first five months, or the first six months from birth, or longer, e.g., when the infant is a full-term infant delivered at about 40 weeks of gestation or later. After the first few months, when the infant is less vulnerable to the effects of severe RSV disease, protection against RSV infection may wane. Thus vital protection is provided during the period when it is most needed. In the case of a pre-term infant, favorably protection is provided for a longer period from birth for example an additional time period at least equaling the time interval between birth of the infant and what would have been 35 weeks gestation (i.e., by about 5 extra weeks), or 38 weeks gestation (by about 2 extra weeks), or longer depending on the gestational age of the infant at birth.

It will be evident that protecting the infant does not necessarily mean 100% protection against infection by RSV. Provided that that there is a reduction in incidence or severity of infection or disease it will be recognized that protection is provided. Protecting the infant favorably includes protecting the infant from severe disease and hospitalization caused by RSV. As such, the methods and uses disclosed herein reduce the incidence or severity of disease caused by RSV such as LRTI or other RSV disease. For example, administration of an immunogenic composition containing an F protein analog as disclosed herein can reduce the incidence (in a cohort of infants of vaccinated mothers) of LRTI by at least about 50%, or at least about 60%, or by 60 to 70%, or by at least about 70%, or by at least about 80%, or by at least about 90% compared to infants of unvaccinated mothers. Favorably, such administration reduces the severity of LRTI by at least about 50%, or at least about 60%, or by 60 to 70%, or by at least about 70%, or by at least about 80%, or by at least about 90% compared to infected infants of unvaccinated mothers. Favorably, such administration reduces the need for hospitalization due to severe RSV disease in such a cohort by at least about 50%, or at least about 60%, or by 60 to 70%, or by at least about 70%, or by at least about 80%, or by at least about 90% compared to infected infants of unvaccinated mothers. Whether there is considered to be a need for hospitalization due to severe LRTI, or whether a particular case of LRTI is hospitalized, may vary from country to country and therefore severe LRTI as judged according to defined clinical symptoms well known in the art may be a better measure than the need for hospitalization.

Typically, the F protein analog is administered to the pregnant female during the third trimester of gestation. The timing of maternal immunization is designed to allow generation of maternal antibodies and transfer of the maternal antibodies to the fetus. Thus, favorably sufficient time elapses between immunization and birth to allow optimum transfer of maternal antibodies across the placenta. Antibody transfer starts in humans generally at about 25 weeks of gestation, increasing up 28 weeks and becoming and remaining optimal from about 30 weeks of gestation. A minimum of about two to four weeks is believed to be needed between maternal immunization as described herein and birth to allow effective transfer of maternal antibodies against RSV F protein to the fetus. Thus maternal immunization can take place any time after 25 weeks of gestation, for example at or after 25, 26, 27, 28, 29, 30, 31, 32, 33 or 34 weeks of gestation (23, 24, 25, 26, 27, 28, 29, 30, 31, 32, 33, 34, 35 or 36 weeks post-conception), or at or before 38, 37, 36, 35, 34, 33, 32, 31 or 30 weeks of gestation (36, 35, 34, 33, 32, 31, 29 or 28 weeks post-conception). Favorably maternal immunization is carried out between 26 and 38 weeks, such as between 28 and 34 weeks of gestation.

Favorably maternal immunization is carried out at least two or at least three or at least four or at least five or at least six weeks prior to the expected date of delivery of the infant. Timing of administration may need to be adjusted in the case of a pregnant female who is at risk of an early delivery, in order to provide sufficient time for generation of antibodies and transfer to the fetus.

Favorably, the F protein analog or formulation thereof is administered to the pregnant female in a single dose, during the period described. Maternal immunization against RSV described herein can be considered as a “booster” for existing maternal immunity against RSV that increases the immune response against RSV that has previously been primed, e.g., by natural exposure to the virus. Thus, it is expected that only a single dose is required. If a second dose is administered, this is favorably also within the time period for administration for the first dose, favorably with a time gap between the first and second doses of for example one to eight weeks or two to six weeks, for example two weeks or four weeks or six weeks.

Administration of the F protein anolog to a pregnant female results in boosting maternal antibody titres, for example, increasing titres of neutralizing antibodies, preferably of the IgG₁ subclass. The increased antibody titre in the mother results in the passive transfer of RSV-specific antibodies with neutralizing effector function to the gestating infant across the placenta via an active transport mechanism mediated by Fc receptors, e.g., in the syncytiotrophoblast of the chorionic villi. Transport across the placenta of RSV-specific IgG₁ antibodies resulting from the immunization methods disclosed herein is expected to be efficient and result in titres, which in infants born at or near term, approach, equal or exceed the titres in maternal circulation. For example, titres of RSV-specific antibodies are favorably at levels of at least 30 mcg/mL at birth. Typically, the titres can be at or above this level, such as at 40 mcg/mL, 50 mcg/mL, 60 mcg/mL, or even higher, such as 75 mcg/mL, 80 mcg/mL, 90 mcg/mL, 100 mcg/mL, or even up to 120 mcg/mL or higher in healthy infants born at full term gestation. These values can be on an individual basis or on a population mean basis. Favorably, the level of antibodies observed at birth is above the stated thresholds and persists for several months following birth.

Neutralizing capacity (neutralization titre) of the transferred antibodies can also be assessed, and provides a measure of functional attribute of the antibodies correlated with protection. A specific quantity of a replication capable RSV virus and a defined dilution of serum are mixed and incubated. The virus-serum reaction mixture is then transferred onto host cells (e.g., HEp-2 cells) permissive for viral replication and incubated under conditions and for a period of time suitable for cell growth and viral replication. Non-neutralized virus is able to infect and replicate in the host cells. This leads to the formation of a given number of plaque forming units (PFU) on the cell monolayer that can be detected using a fluorochrome-tagged anti-RSV antibody. The neutralising titre is determined by calculating the serum dilution inducing a specified level of inhibition (e.g., 50% inhibition or 60% inhibition) in PFUs compared to a cell monolayer infected with virus alone, without serum. For example, the Palivizumab antibody has been shown to have a neutralization titer (50% effective concentration [EC50]) expressed as the antibody concentration required to reduce detection of RSV antigen by 50% compared with untreated virus-infected cells of 0.65 mcg per mL (mean 0.75±0.53 mcg per mL; n=69, range 0.07-2.89 mcg per mL) and 0.28 mcg per mL (mean 0.35±0.23 mcg per mL; n=35, range 0.03-0.88 mcg per mL) against clinical RSV A and RSV B isolates, respectively. Thus, in certain embodiments, the neutralization titre of antibodies transferred via the placenta to the gestational infant can be measured in the infant following birth and has (on a population median basis) an EC50 of at least about 0.50 mcg/mL (for example, at least about 0.65 mcg/mL), or greater for an RSV A strain and an EC50 of at least about 0.3 mcg/mL (for example, at least about 0.35 mcg/mL), or greater for an RSV B strain. Favorably, the neutralizing antibody titre remains above the stated threshold for several weeks to months following birth.

Optionally, in order to extend protection against RSV infection and disease beyond the early months of life during which the passively transferred maternal antibodies provide protection, the infant can be actively immunized to elicit an a adaptive immune response specific for RSV. The composition can comprise an F protein analog, optionally formulated with an adjuvant to enhance the immune response elicited by the antigen. For administration to an infant that has not been previously exposed to RSV, the F protein analog can be formulated with an adjuvant that elicits immune response that is characterized by the production of T cells that exhibit a Th1 cytokine profile (or that is characterized by a balance of T cells that exhibit Th1 and Th2 cytokine profiles).

Alternatively, rather than administering a F protein analog or other protein subunit vaccine, the composition that elicits an adaptive immune response to protect against RSV can include a live attenuated virus vaccine, or a nucleic acid that encodes one or more RSV antigens (such as an F antigen, a G antigen, an N antigen, or a M2 antigen, or portions thereof). For example, the nucleic acid may be in a vector, such as a recombinant viral vector, for example, an adenovirus vector, an adeno-associated virus vector, an MVA vector, a measles vector, or the like. Exemplary viral vectors are disclosed in WO2012 089231, which is incorporated herein for the purpose of illustrating immunogenic compositions that contain a viral vector that encodes one or more RSV antigens. Alternatively, the nucleic acid can be a self replicating nucleic acid, such as a self-replicating RNA, e.g., in the form of a viral replicon, such as an alphavirus replicon (e.g., in the form of a virus replicon particle packaged with virus structural proteins). Examples of such self-replicating RNA replicons are described in WO2012/103361, which is incorporated herein for the purpose of disclosing RNA replicons that encode RSV proteins and their formulation as immunogenic compositions.

When a composition that elicits an adaptive RSV immune response is administered to an infant born to a mother that received an RSV vaccine as disclosed herein during pregnancy, the composition can be administered one or more times. The first administration can be at or near the time of birth (e.g., on the day of or the day following birth), or within 1 week of birth or within about 2 weeks of birth. Alternatively, the first administration can be at about 4 weeks after birth, about 6 weeks after birth, about 2 months after birth, about 3 months after birth, about 4 months after birth, or later, such as about 6 months after birth, about 9 months after birth, or about 12 months after birth. Typically, the first administration wiThus, in an embodiment, this disclosure provides methods for protecting an infant from disease caused by RSV, by administering a composition that elicits an immune response specific for RSV to an infant born to a female to whom an immunogenic composition comprising an F protein analog was administered during the time that she was pregnant with the infant.

### RSV F Protein Analogs

The recombinant RSV antigens disclosed herein are F protein analogs derived from (that is, corresponding immunologically in whole or in part to) the RSV F protein. They can include one or more modifications that alter the structure or function of the F protein but retain the immunological properties of the F protein such that an immune response generated against an F protein analog will recognize the native F protein and thus recognize RSV. F protein analogs described herein are useful as immunogens.

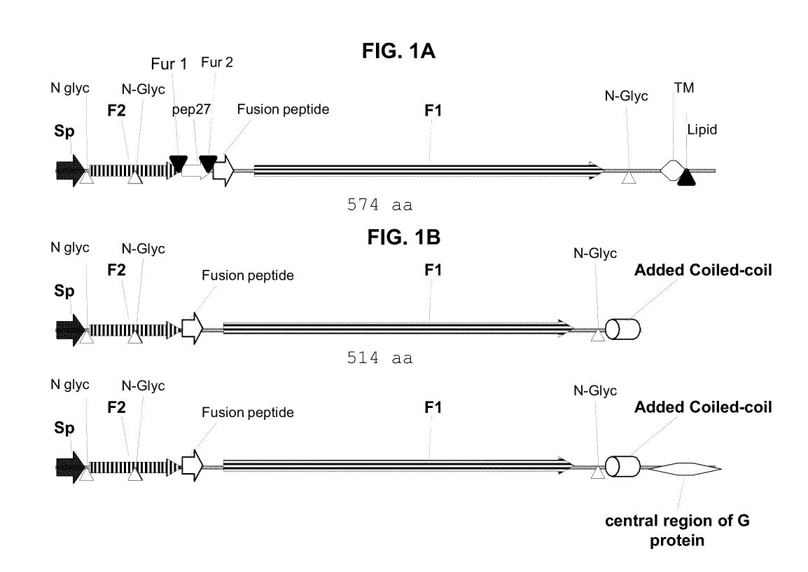

Details of the structure of the RSV F protein are provided herein with reference to terminology and designations widely accepted in the art, and illustrated schematically in FIG. 1A. With reference to the primary amino acid sequence of the F protein polypeptide (FIG. 1A), the following terms are utilized to describe structural features of the F protein analogs.

The term F0 refers to a full-length translated F protein precursor. The F0 polypeptide can be subdivided into an F2 domain and an F1 domain separated by an intervening peptide, designated pep27. During maturation, the F0 polypeptide undergoes proteolytic cleavage at two furin sites situated between F2 and F1 and flanking pep27. For purpose of the ensuing discussion, an F2 domain includes at least a portion, and as much as all, of amino acids 1-109, and a soluble portion of an F1 domain includes at least a portion, and up to all, of amino acids 137-526 of the F protein. As indicated above, these amino acid positions (and all subsequent amino acid positions designated herein) are given in reference to the exemplary F protein precursor polypeptide (F0) of SEQ ID NO:2.

In nature, the RSV F protein is expressed as a single polypeptide precursor 574 amino acids in length, designated F0. In vivo, F0 oligomerizes in the endoplasmic reticulum and is proteolytically processed by a furin protease at two conserved furin consensus sequences (furin cleavage sites), RARR¹⁰⁹ (SEQ ID NO:15) and RKRR¹³⁶ (SEQ ID NO:16) to generate an oligomer consisting of two disulfide-linked fragments. The smaller of these fragments is termed F2 and originates from the N-terminal portion of the F0 precursor. It will be recognized by those of skill in the art that the abbreviations F0, F1 and F2 are commonly designated F₀, F₁ and F₂ in the scientific literature. The larger, C-terminal F1 fragment anchors the F protein in the membrane via a sequence of hydrophobic amino acids, which are adjacent to a 24 amino acid cytoplasmic tail. Three F2-F1 dimers associate to form a mature F protein, which adopts a metastable prefusogenic (“prefusion”) conformation that is triggered to undergo a conformational change upon contact with a target cell membrane. This conformational change exposes a hydrophobic sequence, known as the fusion peptide, which associates with the host cell membrane and promotes fusion of the membrane of the virus, or an infected cell, with the target cell membrane.

The F1 fragment contains at least two heptad repeat domains, designated HRA and HRB, and situated in proximity to the fusion peptide and transmembrane anchor domains, respectively. In the prefusion conformation, the F2-F1 dimer forms a globular head and stalk structure, in which the HRA domains are in a segmented (extended) conformation in the globular head. In contrast, the HRB domains form a three-stranded coiled coil stalk extending from the head region. During transition from the prefusion to the postfusion conformations, the HRA domains collapse and are brought into proximity to the HRB domains to form an anti-parallel six helix bundle. In the postfusion state the fusion peptide and transmembrane domains are juxtaposed to facilitate membrane fusion.

Although the conformational description provided above is based on molecular modeling of crystallographic data, the structural distinctions between the prefusion and postfusion conformations can be monitored without resort to crystallography. For example, electron micrography can be used to distinguish between the prefusion and postfusion (alternatively designated prefusogenic and fusogenic) conformations, as demonstrated by Calder et al., Virology, 271:122-131 (2000) and Morton et al., Virology, 311:275-288, which are incorporated herein by reference for the purpose of their technological teachings. The prefusion conformation can also be distinguished from the fusogenic (postfusion) conformation by liposome association assays as described by Connolly et al., Proc. Natl. Acad. Sci. USA, 103:17903-17908 (2006), which is also incorporated herein by reference for the purpose of its technological teachings. Additionally, prefusion and fusogenic conformations can be distinguished using antibodies that specifically recognize conformation epitopes present on one or the other of the prefusion or fusogenic form of the RSV F protein, but not on the other form. Such conformation epitopes can be due to preferential exposure of an antigenic determinant on the surface of the molecule. Alternatively, conformational epitopes can arise from the juxtaposition of amino acids that are non-contiguous in the linear polypeptide.

Typically, the F protein analogs (PreF, PostF, etc.) analogs lack a transmembrane domain and cytoplasmic tail, and can also be referred to as an F protein ectodomain or soluble F protein ectodomain.

F protein analogs include an F protein polypeptide, which has been modified to stabilize the prefusion conformation of the F protein, that is, the conformation of the mature assembled F protein prior to fusion with the host cell membrane. These F protein analogs are designated “PreF analogs”, “PreF” or “PreF antigens”, for purpose of clarity and simplicity, and are generally soluble. The PreF analogs disclosed herein are predicated on the discovery that soluble F protein analogs that have been modified by the incorporation of a heterologous trimerization domain exhibit improved immunogenic characteristics, and are safe and highly protective when administered to a subject in vivo. Exemplary PreF antigens are described in WO2010149745, herein incorporated by reference in its entirety for the purpose of providing examples of PreF antigens.

F protein analogs also include an F protein polypeptide which has the conformation of the postfusion F protein and which may be referred to as a PostF antigen or postfusion antigen. PostF analogs are described in WO2011008974, incorporated herein by reference. The PostF antigen contains at least one modification to alter the structure or function of the native postfusion F protein.

In a preferred embodiment, the composition for maternal immunization to protect an infant against RSV disease includes an RSV PreF analog antigen that is stabilized in the prefusion conformation found on the virus prior to cellular adhesion and fusion. A schematic illustration of exemplary PreF analogs is provided in FIG. 1B. It will be understood by those of skill in the art that any RSV F protein can be modified to stabilize the prefusion conformation according to the teachings provided herein. Therefore, to facilitate understanding of the principles guiding production of PreF (as well as PostF, and other conformational) analogs, individual structural components will be indicated with reference to an exemplary F protein, the polynucleotide and amino acid sequence of which are provided in SEQ ID NOs:1 and 2, respectively. Similarly, where applicable, G protein antigens are described in reference to an exemplary G protein, the polynucleotide and amino acid sequences of which are provided in SEQ ID NOs:3 and 4, respectively.

The PreF analogs disclosed herein are designed to stabilize and maintain the prefusion conformation of the RSV F protein, such that in a population of expressed protein, a substantial portion of the population of expressed protein is in the prefusogenic (prefusion) conformation (e.g., as predicted by structural and/or thermodynamic modeling or as assessed by one or more of the methods disclosed above). Stabilizing modifications are introduced into a native (or synthetic) F protein, such as the exemplary F protein of SEQ ID NO:2, such that the major immunogenic epitopes of the prefusion conformation of the F protein are maintained following introduction of the PreF analog into a cellular or extracellular environment (for example, in vivo, e.g., following administration to a subject).

First, a heterologous stabilizing domain can be placed at the C-terminal end of the construct in order to replace the membrane anchoring domain of the F0 polypeptide. This stabilizing domain is predicted to compensate for the HRB instability, helping to stabilize the—prefusion conformer. In exemplary embodiments, the heterologous stabilizing domain is a protein multimerization domain. One particularly favorable example of such a protein multimerization domain is a trimerization domain. Exemplary trimerization domains fold into a coiled-coil that promotes assembly into trimers of multiple polypeptides having such coiled-coil domains. Examples of trimerization domains include trimerization domains from influenza hemagglutinin, SARS spike, HIV gp41, modified GCN4, bacteriophage T4 fibritin and ATCase. One favorable example of a trimerization domain is an isoleucine zipper. An exemplary isoleucine zipper domain is the engineered yeast GCN4 isoleucine variant described by Harbury et al. Science 262:1401-1407 (1993). The sequence of one suitable isoleucine zipper domain is represented by SEQ ID NO:11, although variants of this sequence that retain the ability to form a coiled-coil stabilizing domain are equally suitable. Alternative stabilizing coiled coil trimerization domains include: TRAF2 (GENBANK® Accession No. Q12933 [gi:23503103]; amino acids 299-348); Thrombospondin 1 (Accession No. PO7996 [gi:135717]; amino acids 291-314); Matrilin-4 (Accession No. O95460 [gi:14548117]; amino acids 594-618; CMP (matrilin-1) (Accession No. NP_{—}002370 [gi:4505111]; amino acids 463-496; HSF1 (Accession No. AAX42211 [gi:61362386]; amino acids 165-191; and Cubilin (Accession No. NP_{—}001072 [gi:4557503]; amino acids 104-138. It is expected that a suitable trimerization domain results in the assembly of a substantial portion of the expressed protein into trimers. For example, at least 50% of a recombinant PreF polypeptide having a trimerization domain will assemble into a trimer (e.g., as assessed by AFF-MALS). Typically, at least 60%, more favorably at least 70%, and most desirably at least about 75% or more of the expressed polypeptide exists as a trimer.

Another example of a stabilizing mutation is the addition or substitution of a hydrophilic amino acid into a hydrophobic domain of the F protein. Typically, a charged amino acid, such as lysine, will be added or substituted for a neutral residue, such as leucine, in the hydrophobic region. For example, a hydrophilic amino acid can be added to, or substituted for, a hydrophobic or neutral amino acid within the HRB coiled-coil domain of the F protein extracellular domain. By way of example, a charged amino acid residue, such as lysine, can be substituted for the leucine present at position 512 the F protein (relative to the native F0 polypeptide; L482K of the exemplary PreF analog polypeptide of SEQ ID NO:6). Alternatively, or in addition, a hydrophilic amino acid can be added to, or substituted for, a hydrophobic or neutral amino acid within the HRA domain of the F protein. For example, one or more charged amino acids, such as lysine, can be inserted at or near position 105-106 (e.g., following the amino acid corresponding to residue 105 of reference SEQ ID NO:2, such as between amino acids 105 and 106) of the PreF analog). Optionally, hydrophilic amino acids can be added or substituted in both the HRA and HRB domains. Alternatively, one or more hydrophobic residues can be deleted, so long as the overall conformation of the PreF analog is not adversely impacted.

Secondly, pep27 can be removed. Analysis of a structural model of the RSV F protein in the prefusion state suggests that pep27 creates a large unconstrained loop between F1 and F2. This loop does not contribute to stabilization of the prefusion state, and is removed following cleavage of the native protein by furin. Thus, pep27 can also be removed from embodiments that involve a postfusion (or other) conformational analog.

Third, one or both furin cleavage motifs can be deleted (from between the F2 and F1 domains in the native F0 protein). One or both furin recognition sites, located at positions 105-109 or 106-109 and at positions 133-136 can be eliminated by deleting or substituting one or more amino acid of the furin recognition sites, for example deleting one or more amino acids or substituting one or more amino acids or a combination of one or more substitutions or deletions, or modifying such that the protease is incapable of cleaving the PreF (or other F protein analog) polypeptide into its constituent domains. Optionally, the intervening pep27 peptide can also be removed or substituted, e.g., by a linker peptide. Additionally, or optionally, a non-furin cleavage site (e.g., a metalloproteinase site at positions 112-113) in proximity to the fusion peptide can be removed or substituted.

Thus, an F protein analog for use in the methods and uses according to the invention can be obtained which is an uncleaved ectodomain having one or more altered furin cleavage sites. Such F protein analog polypeptides are produced recombinantly in a host cell which secretes them uncleaved at position from amino acid 101 to 161, e.g. not cleaved at the furin cleavage sites at positions 105-109 and 131-136. In particular embodiments, the substitution K131Q, the deletion of the amino acids at positions 131-134, or the substitutions K131Q or R133Q or R135Q or R136Q, are used to inhibit cleavage at 136/137.

In an exemplary design, the fusion peptide is not cleaved from F2, preventing release from the globular head of the prefusion conformer and accessibility to nearby membranes. Interaction between the fusion peptide and the membrane interface is predicted to be a major issue in the prefusion state instability. During the fusion process, interaction between the fusion peptide and the target membrane results in the exposure of the fusion peptide from within the globular head structure, enhancing instability of the prefusion state and folding into post-fusion conformer. This conformation change enables the process of membrane fusion. Removal of one or both of the furin cleavage sites is predicted to prevent membrane accessibility to the N-terminal part of the fusion peptide, stabilizing the prefusion state. Thus, in exemplary embodiments disclosed herein, removal of the furin cleavage motifs results in a PreF analog that comprises an intact fusion peptide, which is not cleaved by furin during or following processing and assembly.

Optionally, at least one non-furin cleavage site can also be removed, for example by substitution of one or more amino acids. For example, experimental evidence suggests that under conditions conducive to cleavage by certain metalloproteinases, the F protein analog can be cleaved in the vicinity of amino acids 110-118 (for example, with cleavage occurring between amino acids 112 and 113 of the F protein analog; between a leucine at position 142 and glycine at position 143 of the reference F protein polypeptide of SEQ ID NO:2). Accordingly, modification of one or more amino acids within this region can reduce cleavage of the F protein analog. For example, the leucine at position 112 can be substituted with a different amino acid, such as isoleucine, glutamine or tryptophan (as shown in the exemplary embodiment of SEQ ID NO:20). Alternatively or additionally, the glycine at position 113 can be substituted by a serine or alanine. In further embodiments the F protein analogs further contain altered trypsin cleavage sites, and F protein analogs are not cleaved by trypsin at a site between amino acid 101 and 161.

Optionally, a F protein analog can include one or more modifications that alters the glycosylation pattern or status (e.g., by increasing or decreasing the proportion of molecules glycosylated at one or more of the glycosylation sites present in a native F protein polypeptide). For example, the native F protein polypeptide of SEQ ID NO:2 is predicted to be glycosylated at amino acid positions 27, 70 and 500 (corresponding to positions 27, 70 and 470 of the exemplary PreF analog of SEQ ID NO:6). In an embodiment, a modification is introduced in the vicinity of the glycosylation site at amino acid position 500 (designated N470). For example, the glycosylation site can be removed by substituting an amino acid, such as glutamine (Q) in place of the asparagine at position 500 (of the reference sequence, which corresponds by alignment to position 470 of the exemplary PreF analog). Favorably, a modification that increases glycosylation efficiency at this glycosylation site is introduced. Examples of suitable modifications include at positions 500-502, the following amino acid sequences: NGS; NKS; NGT; NKT. Interestingly, it has been found that modifications of this glycosylation site that result in increased glycosylation also result in substantially increased PreF production. Thus, in certain embodiments, the PreF analogs have a modified glycosylation site at the position corresponding to amino acid 500 of the reference PreF sequence (SEQ ID NO:2), e.g., at position 470 of the PreF analog exemplified by SEQ ID NO:6). Suitable, modifications include the sequences: NGS; NKS; NGT; NKT at amino acids corresponding to positions 500-502 of the reference F protein polypeptide sequence. The amino acid of an exemplary embodiment that includes an “NGT” modification is provided in SEQ ID NO:18. One of skill in the art can easily determine similar modifications for corresponding NGS, NKS, and NKT modifications. Such modifications are favorably combined with any of the stabilizing mutations disclosed herein (e.g., a heterologous coiled-coil, such as an isoleucine zipper, domain and/or a modification in a hydrophobic region, and/or removal of pep27, and/or removal of a furin cleavage site, and/or removal of a non-furin cleavage site, and/or removal of a non-furin cleavage site). For example, in one specific embodiment, the F protein analog includes a substitution that eliminates a non-furin cleavage site and a modification that increases glycosylation. An exemplary PreF analog sequence is provided in SEQ ID NO:22 (which exemplary embodiment includes an “NGT” modification and the substitution of glutamine in the place of leucine at position 112). For example, in certain exemplary embodiments, the glycosylation modified PreF analogs are selected from the group of: a) a polypeptide comprising or consisting of SEQ ID NO:22; b) a polypeptide encoded by SEQ ID NO:21 or by a polynucleotide sequence that hybridizes under stringent conditions over substantially its entire length to SEQ ID NO:21; c) a polypeptide with at least 95% sequence identity to SEQ ID NO:22.

More generally, any one of the stabilizing modifications disclosed herein, e.g., addition of a heterologous stabilizing domain, such as a coiled-coil (for example, an isoleucine zipper domain), preferably situated at the C-terminal end of the soluble F protein analog; modification of a residue, such as leucine to lysine, in the hydrophobic HRB domain; removal of pep27; removal of one or both furin cleavage motifs; removal of a non-furin cleavage site such as a trypsin cleavage site; and/or modification of a glycosylation site can be employed in combination with any one or more (or up to all-in any desired combination) of the other stabilizing modifications. For example, a heterologous coiled-coil (or other heterologous stabilizing domain) can be utilized alone or in combination with any of: a modification in a hydrophobic region, and/or removal of pep27, and/or removal of a furin cleavage site, and/or removal of a non-furin cleavage site, and/or removal of a non-furin cleavage site. In certain specific embodiments, the F protein analog, such as the PreF analog, includes a C-terminal coiled-coil (isoleucine zipper) domain, a stabilizing substitution in the HRB hydrophobic domain, and removal of one or both furin cleavage sites. Such an embodiment includes an intact fusion peptide that is not removed by furin cleavage. In one specific embodiment, the F protein analog also includes a modified glycosylation site at amino acid position 500.

The F protein analog for the methods and uses described herein can be produced by a method which comprises providing a biological material containing the F protein analog (e.g., PreF analog, PostF analog or uncleaved F protein ectodomain, etc.) and purifying the analog polypeptide monomers or multimers (e.g., trimers) or a mixture thereof from the biological material.

The F protein analog can be in the form of polypeptide monomers or trimers, or a mixture of monomers and trimers which may exist in equilibrium. The presence of a single form may provide advantages such as a more predictable immune response and better stability.

Thus, in an embodiment, the F protein analog for use in the invention is a purified F protein analog, which may be in the form of monomers or trimers or a mixture of monomers and trimers, substantially free of lipids and lipoproteins.

The F protein polypeptide can be selected from any F protein of an RSV A or RSV B strain, or from variants thereof (as defined above). In certain exemplary embodiments, the F protein polypeptide is the F protein represented by SEQ ID NO:2. To facilitate understanding of this disclosure, all amino acid residue positions, regardless of strain, are given with respect to (that is, the amino acid residue position corresponds to) the amino acid position of the exemplary F protein. Comparable amino acid positions of any other RSV A or B strain can be determined easily by those of ordinary skill in the art by aligning the amino acid sequences of the selected RSV strain with that of the exemplary sequence using readily available and well-known alignment algorithms (such as BLAST, e.g., using default parameters). Numerous additional examples of F protein polypeptides from different RSV strains are disclosed in WO2008114149 (which is incorporated herein by reference for the purpose of providing additional examples of RSV F and G protein sequences). Additional variants can arise through genetic drift, or can be produced artificially using site directed or random mutagenesis, or by recombination of two or more preexisting variants. Such additional variants are also suitable in the context of the F protein analogs utilized in the context of the immunization methods disclosed herein.

In alternative embodiments useful in the methods and uses described herein the recombinant RSV protein is an F protein analog as described in WO2011008974, incorporated herein by reference for the purpose of describing additional F protein analogs, see for example F protein analogs in FIG. 1 of WO2011008974 and also described in Example 1 of WO2011008974.

In selecting F2 and F1 domains of the F protein, one of skill in the art will recognize that it is not strictly necessary to include the entire F2 and/or F1 domain. Typically, conformational considerations are of importance when selecting a subsequence (or fragment) of the F2 domain. Thus, the F2 domain typically includes a portion of the F2 domain that facilitates assembly and stability of the polypeptide. In certain exemplary variants, the F2 domain includes amino acids 26-105. However, variants having minor modifications in length (by addition, or deletion of one or more amino acids) are also possible.

Typically, at least a subsequence (or fragment) of the F1 domain is selected and designed to maintain a stable conformation that includes immunodominant epitopes of the F protein. For example, it is generally desirable to select a subsequence of the F1 polypeptide domain that includes epitopes recognized by neutralizing antibodies in the regions of amino acids 262-275 (palivizumab neutralization) and 423-436 (Centocor's ch101F MAb). Additionally, desirable to include T cell epitopes, e.g., in the region of animo acids 328-355. Most commonly, as a single contiguous portion of the F1 subunit (e.g., spanning amino acids 262-436) but epitopes could be retained in a synthetic sequence that includes these immunodominant epitopes as discontinuous elements assembled in a stable conformation. Thus, an F1 domain polypeptide comprises at least about amino acids 262-436 of an RSV F protein polypeptide. In one non-limiting example provided herein, the F1 domain comprises amino acids 137 to 516 of a native F protein polypeptide. One of skill in the art will recognize that additional shorter subsequences can be used at the discretion of the practitioner.

When selecting a subsequence of the F2 or F1 domain (e.g., as discussed below with respect to the G protein component of certain PreF-G analogs), in addition to conformational consideration, it can be desirable to choose sequences (e.g., variants, subsequences, and the like) based on the inclusion of additional immunogenic epitopes. For example, additional T cell epitopes can be identified using anchor motifs or other methods, such as neural net or polynomial determinations, known in the art, see, e.g., RANKPEP (available on the world wide web at: mif.dfci.harvard.edu/Tools/rankpep.html); ProPredI (available on the world wide web at: imtech.res.in/raghava/propredFindex.html); Bimas (available on the world wide web at: www-bimas.dcrt.nih.gov/molbi/hla_bind/index.html); and SYFPEITH (available on the world wide web at: syfpeithi.bmi-heidelberg.com/scripts/MHCServer.dll/home.htm). For example, algorithms are used to determine the “binding threshold” of peptides, and to select those with scores that give them a high probability of MHC or antibody binding at a certain affinity. The algorithms are based either on the effects on MHC binding of a particular amino acid at a particular position, the effects on antibody binding of a particular amino acid at a particular position, or the effects on binding of a particular substitution in a motif-containing peptide. Within the context of an immunogenic peptide, a “conserved residue” is one which appears in a significantly higher frequency than would be expected by random distribution at a particular position in a peptide. Anchor residues are conserved residues that provide a contact point with the MHC molecule. T cell epitopes identified by such predictive methods can be confirmed by measuring their binding to a specific MHC protein and by their ability to stimulate T cells when presented in the context of the MHC protein.

Favorably, the an F protein analog, for example a PreF analogs (including PreF-G analogs as discussed below), a Post F analog, or other conformational analog, include a signal peptide corresponding to the expression system, for example, a mammalian or viral signal peptide, such as an RSV F0 native signal sequence (e.g., amino acids 1-25 of SEQ ID NO:2 or amino acids 1-25 of SEQ ID NO:6). Typically, the signal peptide is selected to be compatible with the cells selected for recombinant expression. For example, a signal peptide (such as a baculovirus signal peptide, or the melittin signal peptide, can be substituted for expression, in insect cells. Suitable plant signal peptides are known in the art, if a plant expression system is preferred. Numerous exemplary signal peptides are known in the art, (see, e.g., see Zhang & Henzel, Protein Sci., 13:2819-2824 (2004), which describes numerous human signal peptides) and are catalogued, e.g., in the SPdb signal peptide database, which includes signal sequences of archaea, prokaryotes and eukaryotes (http://proline.bic.nus.edu.sg/spdb/). Optionally, any of the preceding antigens can include an additional sequence or tag, such as a His-tag to facilitate purification.

Optionally, the F protein analog (for example, the PreF or Post F or other analog) can include additional immunogenic components. In certain particularly favorable embodiments, the F protein analog includes an RSV G protein antigenic component. Exemplary chimeric proteins having a PreF and G component include the following PreF_V1 (represented by SEQ ID NOs:7 and 8) and PreF_V2 (represented by SEQ ID NOs:9 and 10).

In the PreF-G analogs, an antigenic portion of the G protein (e.g., a truncated G protein, such as amino acid residues 149-229) is added at the C-terminal end of the construct. Typically, the G protein component is joined to the F protein component via a flexible linker sequence. For example, in the exemplary PreF_V1 design, the G protein is joined to the PreF component by a -GGSGGSGGS-linker (SEQ ID NO:14). In the PreF_V2 design, the linker is shorter. Instead of having the -GGSGGSGGS-linker (SEQ ID NO:14), PreF_V2 has 2 glycines (-GG-) for linker

Where present, the G protein polypeptide domain can include all or part of a G protein selected from any RSV A or RSV B strain. In certain exemplary embodiments, the G protein is (or is 95% identical to) the G protein represented by SEQ ID NO:4. Additional examples of suitable G protein sequences can be found in WO2008114149 (which is incorporated herein by reference).

The G protein polypeptide component is selected to include at least a subsequence (or fragment) of the G protein that retains the immunodominant T cell epitope(s), e.g., in the region of amino acids 183-197, such as fragments of the G protein that include amino acids 151-229, 149-229, or 128-229 of a native G protein. In one exemplary embodiment, the G protein polypeptide is a subsequence (or fragment) of a native G protein polypeptide that includes all or part of amino acid residues 149 to 229 of a native G protein polypeptide. One of skill in the art will readily appreciate that longer or shorter portions of the G protein can also be used, so long as the portion selected does not conformationally destabilize or disrupt expression, folding or processing of the F protein analog. Optionally, the G protein domain includes an amino acid substitution at position 191, which has previously been shown to be involved in reducing and/or preventing enhanced disease characterized by eosinophilia associated with formalin inactivated RSV vaccines. A thorough description of the attributes of naturally occurring and substituted (N191A) G proteins can be found, e.g., in US Patent Publication No. 2005/0042230, which is incorporated herein by reference.

Alternatively, the F protein analog can be formulated in an immunogenic composition that also contains a second polypeptide that includes a G protein component. The G protein component typically includes at least amino acids 149-229 of a G protein. Although smaller portions of the G protein can be used, such fragments should include, at a minimum, the immunological dominant epitope of amino acids 184-198. Alternatively, the G protein can include a larger portion of the G protein, such as amino acids 128-229 or 130-230, optionally as an element of a larger protein, such as a full-length G protein, or a chimeric polypeptide.

For example, with respect to selection of sequences corresponding to naturally occurring strains, one or more of the domains can correspond in sequence to an RSV A or B strain, such as the common laboratory isolates designated A2 or Long, or any other naturally occurring strain or isolate. Numerous strains of RSV have been isolated to date. Exemplary strains indicated by GenBank and/or EMBL Accession number can be found in WO2008114149, which is incorporated herein by reference for the purpose of disclosing the nucleic acid and polypeptide sequences of RSV F suitable for use in F protein analogs disclosed herein. Additional strains of RSV are likely to be isolated, and are encompassed within the genus of RSV. Similarly, the genus of RSV encompasses variants arising from naturally occurring (e.g., previously or subsequently identified strains) by genetic drift, and/or recombination.

In addition to such naturally occurring and isolated variants, engineered variants that share sequence similarity with the aforementioned sequences can also be employed in the context of F protein analogs, including PreF, PostF or other analogs (including F-G) analogs. It will be understood by those of skill in the art, that the similarity between F protein analog polypeptide (and polynucleotide sequences as described below), as for polypeptide (and nucleotide sequences in general), can be expressed in terms of the similarity between the sequences, otherwise referred to as sequence identity. Sequence identity is frequently measured in terms of percentage identity (or similarity); the higher the percentage, the more similar are the primary structures of the two sequences. In general, the more similar the primary structures of two amino acid (or polynucleotide) sequences, the more similar are the higher order structures resulting from folding and assembly. Variants of an F protein, polypeptide (and polynucleotide) sequences typically have one or a small number of amino acid deletions, additions or substitutions but will nonetheless share a very high percentage of their amino acid, and generally their polynucleotide sequence. More importantly, the variants retain the structural and, thus, conformational attributes of the reference sequences disclosed herein.

Methods of determining sequence identity are well known in the art, and are applicable to F protein analog polypeptides, as well as the nucleic acids that encode them (e.g., as described below). Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations. The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

In some instances, the F protein analog has one or more amino acid modifications relative to the amino acid sequence of the naturally occurring strain from which it is derived (e.g., in addition to the aforementioned stabilizing modifications). Such differences can be an addition, deletion or substitution of one or more amino acids. A variant typically differs by no more than about 1%, or 2%, or 5%, or 10%, or 15%, or 20% of the amino acid residues. For example, a variant F protein analog, e.g., PreF or PostF or other analog polypeptide sequence can include 1, or 2, or up to 5, or up to about 10, or up to about 15, or up to about 50, or up to about 100 amino acid differences as compared to the relevant portion of a reference F protein sequence (for example, the PreF analog polypeptide sequences of SEQ ID NOs:6, 8, 10, 18, 20 and/or 22. Thus, a variant in the context of an RSV F or G protein, or F protein analog, typically shares at least 80%, or 85%, more commonly, at least about 90% or more, such as 95%, or even 98% or 99% sequence identity with a reference protein, e.g., in the case of a PreF analog: the reference sequences illustrated in SEQ ID NO:2, 4, 6, 8, 10, 18, and/or 22, or any of the exemplary PreF analogs disclosed herein. Additional variants included as a feature of this disclosure are F protein analogs that include all or part of a nucleotide or amino acid sequence selected from the naturally occurring variants disclosed in WO2008114149. Additional variants can arise through genetic drift, or can be produced artificially using site directed or random mutagenesis, or by recombination of two or more preexisting variants. Such additional variants are also suitable in the context of the F protein analog antigens disclosed herein. For example, the modification can be a substitution of one or more amino acids (such as two amino acids, three amino acids, four amino acids, five amino acids, up to about ten amino acids, or more) that do not alter the conformation or immunogenic epitopes of the resulting F protein analog.

Alternatively or additionally, the modification can include a deletion of one or more amino acids and/or an addition of one or more amino acids. Indeed, if desired, one or more of the polypeptide domains can be a synthetic polypeptide that does not correspond to any single strain, but includes component subsequences from multiple strains, or even from a consensus sequence deduced by aligning multiple strains of RSV virus polypeptides. In certain embodiments, one or more of the polypeptide domains is modified by the addition of an amino acid sequence that constitutes a tag, which facilitates subsequent processing or purification. Such a tag can be an antigenic or epitope tag, an enzymatic tag or a polyhistidine tag. Typically the tag is situated at one or the other end of the protein, such as at the C-terminus or N-terminus of the antigen or fusion protein.

The F protein analogs (and also where applicable, G antigens) disclosed herein can be produced using well established procedures for the expression and purification of recombinant proteins.

In brief, recombinant nucleic acids that encode the F protein analogs are introduced into host cells by any of a variety of well-known procedures, such as electroporation, liposome mediated transfection, Calcium phosphate precipitation, infection, transfection and the like, depending on the selection of vectors and host cells. Favorable host cells include prokaryotic (i.e., bacterial) host cells, such as E. coli, as well as numerous eukaryotic host cells, including fungal (e.g., yeast, such as Saccharomyces cerevisiae and Picchia pastoris) cells, insect cells, plant cells, and mammalian cells (such as 3T3, COS, CHO, BHK, HEK 293) or Bowes melanoma cells. Following expression in a selected host cell, the recombinant F protein analogs can be isolated and/or purified according to procedures well-known in the art. Exemplary expression methods, as well as nucleic acids that encode PreF analogs (including PreF-G analogs) are provided in WO2010149745, which is incorporated herein for the purpose of providing suitable methods for the expression and purification of F protein analogs.

### Immunogenic Compositions

Following expression and purification, the F protein analogs are typically formulated into immunogenic compositions for administration to a pregnant female, and where desired into formulations for administration to the infant following birth. Such formulations typically contain a pharmaceutically acceptable carrier or excipient. Optionally, additional antigens can also be included in the formulation, such as another RSV antigen (e.g., a G protein antigen as described in WO2010149745) or a human metapneumovirus (hMPV) antigen, or a pertussis antigen, or an influenza antigen. WO2010149743 describes examples of hMPV antigens that can be combined with RSV antigens, and is incorporated herein by reference for the purpose of providing examples of hMPV antigens. Gall S. A. et al. Maternal Immunization with tetanus-diphtheria-pertussis vaccine: effect on maternal and neonatal serum antibody levels. Am J Obstet Gynecol 2011:204, describes immunization of pregnant mothers with diphtheria-tetanus-pertussis (Tdap) vaccine containing pertussis antigens pertactin (PRN), pertussis toxin (PT), filamentous hemagglutinin (FHA) and fimbriae (FIM) 2/3 and is incorporated herein by reference for the purpose of providing additional details on maternal immunization using pertussis antigens.

Pharmaceutically acceptable carriers and excipients are well known and can be selected by those of skill in the art. For example, the carrier or excipient can favorably include a buffer. Optionally, the carrier or excipient also contains at least one component that stabilizes solubility and/or stability. Examples of solubilizing/stabilizing agents include detergents, for example, laurel sarcosine and/or tween. Alternative solubilizing/stabilizing agents include arginine, and glass forming polyols (such as sucrose, trehalose and the like). Numerous pharmaceutically acceptable carriers and/or pharmaceutically acceptable excipients are known in the art and are described, e.g., in Remington's Pharmaceutical Sciences, by E. W. Martin, Mack Publishing Co., Easton, Pa., 5th Edition (975).

Accordingly, suitable excipients and carriers can be selected by those of skill in the art to produce a formulation suitable for delivery to a subject by a selected route of administration.

Suitable excipients include, without limitation: glycerol, Polyethylene glycol (PEG), Sorbitol, Trehalose, N-lauroylsarcosine sodium salt, L-proline, Non detergent sulfobetaine, Guanidine hydrochloride, Urea, Trimethylamine oxide, KCl, Cat²⁺, Mg²⁺, Mn²⁺, Zn²⁺ and other divalent cation related salts, Dithiothreitol, Dithioerytrol, and β-mercaptoethanol. Other excipients can be detergents (including: Tween80, Tween20, Triton X-00, NP-40, Empigen BB, Octylglucoside, Lauroyl maltoside, Zwittergent 3-08, Zwittergent 3-0, Zwittergent 3-2, Zwittergent 3-4, Zwittergent 3-6, CHAPS, Sodium deoxycholate, Sodium dodecyl sulphate, Cetyltrimethylammonium bromide).

In compositions containing an F protein analog for the methods and uses described herein, the composition is designed to induce a strong neutralizing antibody response. Mothers have already been exposed to RSV and therefore will have an existing primed response, so the goal for providing protection for the infant is to boost this primed response as effectively as possible and in respect of the antibody subclasses such as IgG₁ that can cross the placenta with high efficiency and provide protection to the infant. This can be achieved without including an adjuvant, or by including an adjuvant that includes only mineral salts such as an aluminium or calcium salts, in particular aluminium hydroxide (alum), aluminium phosphate or calcium phosphate. Alternatively, this can be achieved by formulating with an oil and water emulsion adjuvant, or another adjuvant that enhances the production of antibodies of the IgG₁ subclass. Thus the F protein analog for the methods and uses described herein is favorably formulated with a mineral salt, favorably alum, or with an oil and water emulsion adjuvant.

Any adjuvant is selected to be safe and well tolerated in pregnant women. Optionally, the immunogenic compositions also include an adjuvant other than alum. For example, adjuvants including one or more of 3D-MPL, squalene (e.g., QS21), liposomes, and/or oil and water emulsions are favorably selected, provided that the final formulation enhances the production in the primed female of RSV-specific antibodies with the desired characteristics (e.g., of subclass and neutralizing function).

In some instances, the adjuvant formulation includes a mineral salt, such as a calcium or aluminium (alum) salt, for example calcium phosphate, aluminium phosphate or aluminium hydroxide. Where alum is present, either alone, or e.g., in combination with 3D-MPL, the amount is typically between about 100 μg and 1 mg, such as from about 100 μg, or about 200 μg to about 750 μg, such as about 500 μg per dose.

In some embodiments, the adjuvant includes an oil and water emulsion, e.g., an oil-in-water emulsion. One example of an oil-in-water emulsion comprises a metabolisable oil, such as squalene, a tocol such as a tocopherol, e.g., alpha-tocopherol, and a surfactant, such as sorbitan trioleate (Span 85™) or polyoxyethylene sorbitan monooleate (Tween 80™), in an aqueous carrier. An example of an oil-in-water emulsion is MF59. In certain embodiments, the oil-in-water emulsion does not contain any additional immunostimulants(s), (in particular it does not contain a non-toxic lipid A derivative, such as 3D-MPL, or a saponin, such as QS21). The aqueous carrier can be, for example, phosphate buffered saline. Additionally the oil-in-water emulsion can contain span 85 and/or lecithin and/or tricaprylin.

In another embodiment of the invention the adjuvant composition comprises an oil-in-water emulsion and optionally one or more further immunostimulants, wherein said oil-in-water emulsion comprises 0.5-10 mg metabolisable oil (suitably squalene), 0.5-11 mg tocol (suitably a tocopherol, such as alpha-tocopherol) and 0.4-4 mg emulsifying agent.

Other adjuvants that can be used in immunogenic compositions with an F protein analog, such as a PreF analog, in the immunogenic compositions for the methods and uses described here, on their own or in combination with 3D-MPL, or another adjuvant described herein, are saponins, such as QS21.

In one embodiment the recombinant F protein analog such as a PreF antigen for the methods and uses described herein is formulated with a saponin for example QS21, in particular a combination of a preF analog and QS21 is provided.

In another embodiment the F protein analog such as PreF antigen for the methods and uses described herein is formulated with QS21 and 3D-MPL.

Saponins are taught in: Lacaille-Dubois, M and Wagner H. (1996. A review of the biological and pharmacological activities of saponins Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit. Rev Ther Drug Carrier Syst, 1996, 12 (1-2):1-55; and EP 0 362 279 B1. Particulate structures, termed Immune Stimulating Complexes (ISCOMS), comprising fractions of Quil A are haemolytic and have been used in the manufacture of vaccines (Morein, B., EP 0 109 942 B1; WO 96/11711; WO 96/33739). The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1, which are incorporated herein by reference. Other saponins which have been used in systemic vaccination studies include those derived from other plant species such as Gypsophila and Saponaria (Bomford et al., Vaccine, 10(9):572-577, 1992).

QS21 is an Hplc purified non-toxic fraction derived from the bark of Quillaja Saponaria Molina. A method for producing QS21 is disclosed in U.S. Pat. No. 5,057,540. Non-reactogenic adjuvant formulations containing QS21 are described in WO 96/33739. The aforementioned references are incorporated by reference herein. Said immunologically active saponin, such as QS21, can be used in amounts of between 1 and 50 μg, per human dose of the immunogenic composition. Advantageously QS21 is used at a level of about 25 μg, for example between 20-30 μg, suitably between 21-29 μg or between 22-28 μg or between 23-27 μg or between 24-26 μg, or 25 μg. In another embodiment, the human dose of the immunogenic composition comprises QS21 at a level of about 10 μg, for example between 5 and 15 μg, suitably between 6-14 μg, for example between 7-13 μg or between 8-12 μg or between 9-11 μg, or 10 μg. In a further embodiment, the human dose of the immunogenic composition comprises QS21 at a level of about 5 μg, for example between 1-9 μg, or between 2-8 μg or suitably between 3-7 μg or 4-6 μg, or 5 μg. Such formulations comprising QS21 and cholesterol have been shown to be successful Th1 stimulating adjuvants when formulated together with an antigen. Thus, for example, PreF polypeptides can favorably be employed in immunogenic compositions with an adjuvant comprising a combination of QS21 and cholesterol.

As indicated above, the adjuvant can include mineral salts such as an aluminium or calcium salts, in particular aluminium hydroxide, aluminium phosphate and calcium phosphate. Such an adjuvant can also include 3D-MPL For example, an adjuvant containing 3D-MPL in combination with an aluminium salt (e.g., aluminium hydroxide or “alum”) is suitable for formulation in an immunogenic composition containing a F protein analog for administration to a human subject.

3D-MPL is a non-toxic bacterial lipopolysaccharide derivative. An example of a suitable non-toxic derivative of lipid A, i.e., monophosphoryl lipid A, or more particularly 3-Deacylated monophoshoryl lipid A (3D-MPL). 3D-MPL is sold under the name MPL by GlaxoSmithKline Biologicals N.A., and is referred throughout the document as MPL or 3D-MPL. See, for example, U.S. Pat. Nos. 4,436,727; 4,877,611; 4,866,034 and 4,912,094. 3D-MPL primarily promotes CD4+ T cell responses with an IFN-γ (Th1) phenotype. 3D-MPL can be produced according to the methods disclosed in GB2220211 A. Chemically it is a mixture of 3-deacylated monophosphoryl lipid A with 3, 4, 5 or 6 acylated chains. In the compositions of the present invention small particle 3D-MPL can be used. Small particle 3D-MPL has a particle size such that it can be sterile-filtered through a 0.22 μm filter. Such preparations are described in WO94/21292.

A lipopolysaccharide, such as 3D-MPL, can be used at amounts between 1 and 50 μg, per human dose of the immunogenic composition. Such 3D-MPL can be used at a level of about 25 μg, for example between 20-30 μg, suitably between 21-29 μg or between 22 and 28 μg or between 23 and 27 μg or between 24 and 26 μg, or 25 μg. In another embodiment, the human dose of the immunogenic composition comprises 3D-MPL at a level of about 10 μg, for example between 5 and 15 μg, suitably between 6 and 14 μg, for example between 7 and 13 μg or between 8 and 12 μg or between 9 and 11 μg, or 10 μg. In a further embodiment, the human dose of the immunogenic composition comprises 3D-MPL at a level of about 5 μg, for example between 1 and 9 μg, or between 2 and 8 μg or suitably between 3 and 7 μg or 4 and μg, or 5 μg.

In other embodiments, the lipopolysaccharide can be a β(1-6) glucosamine disaccharide, as described in U.S. Pat. No. 6,005,099 and EP Patent No. 0 729 473 B1. One of skill in the art would be readily able to produce various lipopolysaccharides, such as 3D-MPL, based on the teachings of these references. Nonetheless, each of these references is incorporated herein by reference. In addition to the aforementioned immunostimulants (that are similar in structure to that of LPS or MPL or 3D-MPL), acylated monosaccharide and disaccharide derivatives that are a sub-portion to the above structure of MPL are also suitable adjuvants. In other embodiments, the adjuvant is a synthetic derivative of lipid A, some of which are described as TLR-4 agonists, and include, but are not limited to: OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026); OM 294 DP (3S,9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO 99/64301 and WO 00/0462); and OM 197 MP-Ac DP (3S-,9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127).

In one specific embodiment, the adjuvant formulation includes 3D-MPL prepared in the form of an emulsion, such as an oil-in-water emulsion. In some cases, the emulsion has a small particle size of less than 0.2 μm in diameter, as disclosed in WO 94/21292. For example, the particles of 3D-MPL can be small enough to be sterile filtered through a 0.22 micron membrane (as described in European Patent number 0 689 454). Alternatively, the 3D-MPL can be prepared in a liposomal formulation. Optionally, the adjuvant containing 3D-MPL (or a derivative thereof) also includes an additional immunostimulatory component.

Combinations of different adjuvants, such as those mentioned hereinabove, can also be used in compositions with F protein analogs such as PreF analogs. For example, as already noted, QS21 can be formulated together with 3D-MPL. The ratio of QS21:3D-MPL will typically be in the order of 1:10 to 10:1; such as 1:5 to 5:1, and often substantially 1:1. Typically, the ratio is in the range of 2.5:1 to 1:1 3D-MPL: QS21. Another combination adjuvant formulation includes 3D-MPL and an aluminium salt, such as aluminium hydroxide.

Other TLR4 ligands which can be used are alkyl Glucosaminide phosphates (AGPs) such as those disclosed in WO 98/50399 or U.S. Pat. No. 6,303,347 (processes for preparation of AGPs are also disclosed), suitably RC527 or RC529 or pharmaceutically acceptable salts of AGPs as disclosed in U.S. Pat. No. 6,764,840. Some AGPs are TLR4 agonists, and some are TLR4 antagonists. Both are thought to be useful as adjuvants.

Other suitable TLR-4 ligands, capable of causing a signaling response through TLR-4 (Sabroe et al, JI 2003 p 1630-5) are, for example, lipopolysaccharide from gram-negative bacteria and its derivatives, or fragments thereof, in particular a non-toxic derivative of LPS (such as 3D-MPL). Other suitable TLR agonists are: heat shock protein (HSP) 10, 60, 65, 70, 75 or 90; surfactant Protein A, hyaluronan oligosaccharides, heparan sulphate fragments, fibronectin fragments, fibrinogen peptides and b-defensin-2, and muramyl dipeptide (MDP). In one embodiment the TLR agonist is HSP 60, 70 or 90. Other suitable TLR-4 ligands are as described in WO 2003/011223 and in WO 2003/099195, such as compound I, compound II and compound III disclosed on pages 4-5 of WO2003/011223 or on pages 3-4 of WO2003/099195 and in particular those compounds disclosed in WO2003/011223 as ER803022, ER803058, ER803732, ER804053, ER804057, ER804058, ER804059, ER804442, ER804680, and ER804764. For example, one suitable TLR-4 ligand is ER804057.

Additional TLR agonists are also useful as adjuvants. The term “TLR agonist” refers to an agent that is capable of causing a signaling response through a TLR signaling pathway, either as a direct ligand or indirectly through generation of endogenous or exogenous ligand. Such natural or synthetic TLR agonists can be used as alternative or additional adjuvants. A brief review of the role of TLRs as adjuvant receptors is provided in Kaisho & Akira, Biochimica et Biophysica Acta 1589:1-13, 2002. These potential adjuvants include, but are not limited to agonists for TLR2, TLR3, TLR7, TLR8 and TLR9. Accordingly, in one embodiment, the adjuvant and immunogenic composition further comprises an adjuvant which is selected from the group consisting of: a TLR-1 agonist, a TLR-2 agonist, TLR-3 agonist, a TLR-4 agonist, TLR-5 agonist, a TLR-6 agonist, TLR-7 agonist, a TLR-8 agonist, TLR-9 agonist, or a combination thereof.

In one embodiment of the present invention, a TLR agonist is used that is capable of causing a signaling response through TLR-1. Suitably, the TLR agonist capable of causing a signaling response through TLR-1 is selected from: Tri-acylated lipopeptides (LPs); phenol-soluble modulin; Mycobacterium tuberculosis LP; S-(2,3-bis(palmitoyloxy)-(2-RS)-propyl)-N-palmitoyl-(R)-Cys-(S)-Ser-(S)-Lys(4)-OH, trihydrochloride (Pam3Cys) LP which mimics the acetylated amino terminus of a bacterial lipoprotein and OspA LP from Borrelia burgdorferi.

In an alternative embodiment, a TLR agonist is used that is capable of causing a signaling response through TLR-2. Suitably, the TLR agonist capable of causing a signaling response through TLR-2 is one or more of a lipoprotein, a peptidoglycan, a bacterial lipopeptide from M. tuberculosis, B. burgdorferi or T. pallidum; peptidoglycans from species including Staphylococcus aureus; lipoteichoic acids, mannuronic acids, Neisseria porins, bacterial fimbriae, Yersina virulence factors, CMV virions, measles haemagglutinin, and zymosan from yeast.

In an alternative embodiment, a TLR agonist is used that is capable of causing a signaling response through TLR-3. Suitably, the TLR agonist capable of causing a signaling response through TLR-3 is double stranded RNA (dsRNA), or polyinosinic-polycytidylic acid (Poly IC), a molecular nucleic acid pattern associated with viral infection.

In an alternative embodiment, a TLR agonist is used that is capable of causing a signaling response through TLR-5. Suitably, the TLR agonist capable of causing a signaling response through TLR-5 is bacterial flagellin.

In an alternative embodiment, a TLR agonist is used that is capable of causing a signaling response through TLR-6. Suitably, the TLR agonist capable of causing a signaling response through TLR-6 is mycobacterial lipoprotein, di-acylated LP, and phenol-soluble modulin. Additional TLR6 agonists are described in WO 2003/043572.

In an alternative embodiment, a TLR agonist is used that is capable of causing a signaling response through TLR-7. Suitably, the TLR agonist capable of causing a signaling response through TLR-7 is a single stranded RNA (ssRNA), loxoribine, a guanosine analogue at positions N7 and C8, or an imidazoquinoline compound, or derivative thereof. In one embodiment, the TLR agonist is imiquimod. Further TLR7 agonists are described in WO 2002/085905.

In an alternative embodiment, a TLR agonist is used that is capable of causing a signaling response through TLR-8. Suitably, the TLR agonist capable of causing a signaling response through TLR-8 is a single stranded RNA (ssRNA), an imidazoquinoline molecule with anti-viral activity, for example resiquimod (R848); resiquimod is also capable of recognition by TLR-7. Other TLR-8 agonists which can be used include those described in WO 2004/071459.

In an alternative embodiment, a TLR agonist is used that is capable of causing a signaling response through TLR-9. In one embodiment, the TLR agonist capable of causing a signaling response through TLR-9 is HSP90. Alternatively, the TLR agonist capable of causing a signaling response through TLR-9 is bacterial or viral DNA, DNA containing unmethylated CpG nucleotides, in particular sequence contexts known as CpG motifs. CpG-containing oligonucleotides induce a predominantly Th1 response. Such oligonucleotides are well known and are described, for example, in WO 96/02555, WO 99/33488 and U.S. Pat. Nos. 6,008,200 and 5,856,462. Suitably, CpG nucleotides are CpG oligonucleotides. Suitable oligonucleotides for use in the immunogenic compositions of the present invention are CpG containing oligonucleotides, optionally containing two or more dinucleotide CpG motifs separated by at least three, suitably at least six or more nucleotides. A CpG motif is a Cytosine nucleotide followed by a Guanine nucleotide. The CpG oligonucleotides of the present invention are typically deoxynucleotides. In a specific embodiment the internucleotide in the oligonucleotide is phosphorodithioate, or suitably a phosphorothioate bond, although phosphodiester and other internucleotide bonds are within the scope of the invention. Also included within the scope of the invention are oligonucleotides with mixed internucleotide linkages. Methods for producing phosphorothioate oligonucleotides or phosphorodithioate are described in U.S. Pat. Nos. 5,666,153, 5,278,302 and WO 95/26204.

Another class of suitable adjuvants for use in formulations with F protein analogs such as PreF analogs includes OMP-based immunostimulatory compositions. OMP-based immunostimulatory compositions are particularly suitable as mucosal adjuvants, e.g., for intranasal administration. OMP-based immunostimulatory compositions are a genus of preparations of outer membrane proteins (OMPs, including some porins) from Gram-negative bacteria, such as, but not limited to, Neisseria species (see, e.g., Lowell et al., J. Exp. Med. 167:658, 1988; Lowell et al., Science 240:800, 1988; Lynch et al., Biophys. J. 45:104, 1984; Lowell, in “New Generation Vaccines” 2nd ed., Marcel Dekker, Inc., New York, Basil, Hong Kong, page 193, 1997; U.S. Pat. No. 5,726,292; U.S. Pat. No. 4,707,543), which are useful as a carrier or in compositions for immunogens, such as bacterial or viral antigens. Some OMP-based immunostimulatory compositions can be referred to as “Proteosomes,” which are hydrophobic and safe for human use. Proteosomes have the capability to auto-assemble into vesicle or vesicle-like OMP clusters of about 20 nm to about 800 nm, and to noncovalently incorporate, coordinate, associate (e.g., electrostatically or hydrophobically), or otherwise cooperate with protein antigens (Ags), particularly antigens that have a hydrophobic moiety. Any preparation method that results in the outer membrane protein component in vesicular or vesicle-like form, including multi-molecular membranous structures or molten globular-like OMP compositions of one or more OMPs, is included within the definition of Proteosome. Proteosomes can be prepared, for example, as described in the art (see, e.g., U.S. Pat. No. 5,726,292 or U.S. Pat. No. 5,985,284). Proteosomes can also contain an endogenous lipopolysaccharide or lipooligosaccharide (LPS or LOS, respectively) originating from the bacteria used to produce the OMP porins (e.g., Neisseria species), which generally will be less than 2% of the total OMP preparation.

Proteosomes are composed primarily of chemically extracted outer membrane proteins (OMPs) from Neisseria menigitidis (mostly porins A and B as well as class 40MP), maintained in solution by detergent (Lowell G H. Proteosomes for Improved Nasal, Oral, or Injectable Vaccines. In: Levine M M, Woodrow G C, Kaper J B, Cobon G S, eds, New Generation Vaccines. New York: Marcel Dekker, Inc. 1997; 193-206). Proteosomes can be formulated with a variety of antigens such as purified or recombinant proteins derived from viral sources, including the F protein analogs such as PreF polypeptides disclosed herein, e.g., by diafiltration or traditional dialysis processes. The gradual removal of detergent allows the formation of particulate hydrophobic complexes of approximately 100-200 nm in diameter (Lowell G H. Proteosomes for Improved Nasal, Oral, or Injectable Vaccines. In: Levine M M, Woodrow G C, Kaper J B, Cobon G S, eds, New Generation Vaccines. New York: Marcel Dekker, Inc. 1997; 193-206).

“Proteosome: LPS or Protollin” as used herein refers to preparations of proteosomes admixed, e.g., by the exogenous addition, with at least one kind of lipo-polysaccharide to provide an OMP-LPS composition (which can function as an immunostimulatory composition). Thus, the OMP-LPS composition can be comprised of two of the basic components of Protollin, which include (1) an outer membrane protein preparation of Proteosomes (e.g., Projuvant) prepared from Gram-negative bacteria, such as Neisseria meningitidis, and (2) a preparation of one or more liposaccharides. A lipo-oligosaccharide can be endogenous (e.g., naturally contained with the OMP Proteosome preparation), can be admixed or combined with an OMP preparation from an exogenously prepared lipo-oligosaccharide (e.g., prepared from a different culture or microorganism than the OMP preparation), or can be a combination thereof. Such exogenously added LPS can be from the same Gram-negative bacterium from which the OMP preparation was made or from a different Gram-negative bacterium. Protollin should also be understood to optionally include lipids, glycolipids, glycoproteins, small molecules, or the like, and combinations thereof. The Protollin can be prepared, for example, as described in U.S. Patent Application Publication No. 2003/0044425.

An immunogenic composition typically contains an immunoprotective quantity (or a fractional dose thereof) of the antigen and can be prepared by conventional techniques. Preparation of immunogenic compositions, including those for administration to human subjects, is generally described in Pharmaceutical Biotechnology, Vol. 61 Vaccine Design—the subunit and adjuvant approach, edited by Powell and Newman, Plenum Press, 1995. New Trends and Developments in Vaccines, edited by Voller et al., University Park Press, Baltimore, Md., U.S.A. 1978. Encapsulation within liposomes is described, for example, by Fullerton, U.S. Pat. No. 4,235,877. Conjugation of proteins to macromolecules is disclosed, for example, by Likhite, U.S. Pat. No. 4,372,945 and by Armor et al., U.S. Pat. No. 4,474,757.

Typically, the amount of protein in each dose of the immunogenic composition is selected as an amount which induces an immunoprotective response without significant, adverse side effects in the typical subject. Immunoprotective in this context does not necessarily mean completely protective against infection; it means protection against symptoms or disease, especially severe disease associated with the virus.

It should be noted that regardless of the adjuvant selected, the concentration in the final formulation is calculated to be safe and effective in the target population of pregnant females.

Additional formulation details can be found in WO2010149745, which is incorporated herein by reference for the purpose of providing additional details concerning formulation of immunogenic compositions comprising F protein analogs such as PreF analogs.

Optionally, the immunogenic compositions containing an F protein analog such as a PreF, or PostF analog are formulated with at least one additional antigen of a pathogenic organism other than RSV. For example, the pathogenic organism can be a pathogen of the respiratory tract (such as a virus or bacterium that causes a respiratory infection). In certain cases, the immunogenic composition contains an antigen derived from a pathogenic virus other than RSV, such as a virus that causes an infection of the respiratory tract, such as influenza or parainfluenza. In other embodiments, the additional antigens are selected to facilitate administration or reduce the number of inoculations required to protect a subject against a plurality of infectious organisms. For example, the antigen can be derived from any one or more of influenza, hepatitis B, diphtheria, tetanus, pertussis, Hemophilus influenza, poliovirus, hMPV, Streptococcus or Pneumococcus, among others. In specific embodiments, the F protein analog such as a PreF analog is formulated with diphtheria, tetanus and pertussis, or with pertussis antigens alone for example all or a selection of PRN, PT, FHA, and FIM2/3, or with one or more pertussis antigens such as one or more of PRN, PT, FHA, and FIM2/3 optionally together with an antigen from another pathogen.

An immunogenic composition for use herein typically contains an immunoprotective quantity (or a fractional dose thereof) of the antigen, or a quantity which provides passive transfer of antibodies so as to be immunoprotective in infants of immunized pregnant females, and can be prepared by conventional techniques. Preparation of immunogenic compositions, including those for administration to human subjects, is generally described in Pharmaceutical Biotechnology, Vol. 61 Vaccine Design—the subunit and adjuvant approach, edited by Powell and Newman, Plenum Press, 1995. New Trends and Developments in Vaccines, edited by Voller et al., University Park Press, Baltimore, Md., U.S.A. 1978. Encapsulation within liposomes is described, for example, by Fullerton, U.S. Pat. No. 4,235,877. Conjugation of proteins to macromolecules is disclosed, for example, by Likhite, U.S. Pat. No. 4,372,945 and by Armor et al., U.S. Pat. No. 4,474,757.

Typically, the amount of protein in each dose of the immunogenic composition is selected as an amount which induces an immunoprotective response in the infant without significant, adverse side effects in the typical subject i.e. the pregnant female or the gestating infant. Immunoprotective in this context does not necessarily mean completely protective against infection; it means protection against symptoms or disease, especially severe disease associated with the virus. The amount of antigen can vary depending upon which specific immunogen is employed. Generally, it is expected that each human dose will comprise 1-1000 μg of protein, such as from about 1 μg to about 100 μg, for example, from about 1 μg to about 60 μg, such as about 1 μg, about 2 μg, about 5 μg, about 10 μg, about 15n, about 20 μg, about 25 μg, about 30 μg, about 40n, about 50 μg, or about 60 μg. Generally a human dose with be in a volume of 0.5 ml. Thus the composition for the uses and methods described herein can be for example 10 μg or 30 μg or 60 μg in a 0.5 ml human dose. The amount utilized in an immunogenic composition is selected based on the subject population. An optimal amount for a particular composition can be ascertained by standard studies involving observation of antibody titres and other responses in subjects. Following an initial vaccination, subjects can receive a boost in about 4-12 weeks, or at any time prior to delivery of the infant, favorably at least two or at least four weeks prior to the expected delivery date.

Maternal immunization as described herein is carried out via a suitable route for administration for an RSV vaccine, including intramuscular, intranasal, or cutaneous administration. Favorably, RSV maternal immunization as described herein is carried out cutaneously, which means that the antigen is introduced into the dermis and/or epidermis of the skin (e.g., intradermally). In a particular embodiment a recombinant RSV antigen comprising an F protein analog such as a PreF antigen or a PostF antigen, is delivered to the pregnant female cutaneously or intradermally. In a particular embodiment the F protein such as PreF antigen or postF antigen is formulated with an adjuvant described herein for example a saponin such as QS21, with or without 3D-MPL, for cutaneous or intradermal delivery. In another embodiment the F protein analog such as PreF or PostF antigen is formulated with a mineral salt such as aluminium hydroxide or aluminium phosphate or calcium phosphate, with or without an immunostimulant such as QS21 or 3D-MPL, for cutaneous or intradermal delivery.

Delivery via the cutaneous route including the intradermal route may require or allow a lower dose of antigen than other routes such as intramuscular delivery. Therefore also provided is an immunogenic composition for cutaneous or intradermal delivery comprising an F protein analog in a low dose e.g. less than the normal intramuscular dose, e.g. 50% or less of the normal intramuscular dose, for example 50 μg or less, or 20 μg or less, or 10 μg or less or 5 μg or less per human dose. Optionally the immunogenic composition for cutaneous or intradermal delivery also comprises an adjuvant e.g. an alum or QS21 or 3D-MPL or a combination thereof.

Devices for cutaneous administration include short needle devices (which have a needle between about 1 and about 2 mm in length) such as those described in U.S. Pat. No. 4,886,499, U.S. Pat. No. 5,190,521, U.S. Pat. No. 5,328,483, U.S. Pat. No. 5,527,288, U.S. Pat. No. 4,270,537, U.S. Pat. No. 5,015,235, U.S. Pat. No. 5,141,496, U.S. Pat. No. 5,417,662 and EP1092444. Cutaneous vaccines may also be administered by devices which limit the effective penetration length of a needle into the skin, such as those described in WO99/34850, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Jet injection devices are described for example in U.S. Pat. No. 5,480,381, U.S. Pat. No. 5,599,302, U.S. Pat. No. 5,334,144, U.S. Pat. No. 5,993,412, U.S. Pat. No. 5,649,912, U.S. Pat. No. 5,569,189,U.S. Pat. No. 5,704,911, U.S. Pat. No. 5,383,851, U.S. Pat. No. 5,893,397, U.S. Pat. No. 5,466,220, U.S. Pat. No. 5,339,163, U.S. Pat. No. 5,312,335, U.S. Pat. No. 5,503,627, U.S. Pat. No. 5,064,413, U.S. Pat. No. 5,520,639, U.S. Pat. No. 4,596,556 U.S. Pat. No. 5 4,790,824,U.S. Pat. No. 4,941,880, U.S. Pat. No. 4,940,460, WO 97/37705 and WO 97/13537.

Devices for cutaneous administration also include ballistic powder/particle delivery devices which use compressed gas to accelerate vaccine in powder form through the outer layers of the skin to the dermis. Additionally, conventional syringes may be used in the classical mantoux method of cutaneous administration. However, the use of conventional syringes requires highly skilled operators and thus devices which are capable of accurate delivery without a highly skilled user are preferred. Additional devices for cutaneous administration include patches comprising immunogenic compositions as described herein. A cutaneous delivery patch will generally comprise a backing plate which includes a solid substrate (e.g. occlusive or nonocclusive surgical dressing). Such patches deliver the immunogenic composition to the dermis or epidermis via microprojections which pierce the stratum corneum. Microprojections are generally between 10 Dm and 2 mm, for example 20 Dm to 500 Dm, 30 Dm to 1 mm, 100 to 200, 200 to 300, 300 to 400, 400 to 500, 500 to 600, 600 to 700, 700, 800, 800 to 900, 100 Dm to 400 Dm, in particular between about 200 Dm and 300 Dm or between about 150 Dm and 250 Dm. Cutaneous delivery patches generally comprise a plurality of microprojections for example between 2 and 5000 microneedles for example between 1000 and 2000 microneedles. The microprojections may be of any shape suitable for piercing the stratum corneum, epidermis and/or dermis Microprojections may be shaped as disclosed in WO2000/074765 and WO2000/074766 for example. The microprojections may have an aspect ratio of at least 3:1 (height to diameter at base), at least about 2:1, or at least about 1:1. A particularly preferred shape for the microprojections is a cone with a polygonal bottom, for example hexagonal or rhombus-shaped. Other possible microprojection shapes are shown, for example, in U.S. Published Patent App. 2004/0087992. In a particular embodiment, microprojections have a shape which becomes thicker towards the base. The number of microprotrusions in the array is preferably at least about 100, at least about 500, at least about 1000, at least about 1400, at least about 1600, or at least about 2000. The area density of microprotrusions, given their small size, may not be particularly high, but for example the number of microprotrusions per cm2 may be at least about 50, at least about 250, at least about 500, at least about 750, at least about 1000, or at least about 1500. In one embodiment of the invention the F protein analog is are delivered to the subject within 5 hours of placing the patch on the skin of the host, for example, within 4 hours, 3 hours, 2 hours, 1 hour or 30 minutes. In a particular embodiment, the F protein analog is delivered within 20 minutes of placing the patch on the skin, for example within 30 seconds 1, 2, 3, 4, 5, 6, 7, 8, 9, 10, 11, 12, 13, 14, 15, 16, 17, 18 or 19 minutes.

The microprojections can be made of any suitable material known to the skilled person. In a particular embodiment at least part of the microprojections are biodegradable, in particular the tip of the microprojection or the outer most layer of the microprojection. In a particular embodiment substantially all the microprojection is biodegradable. The term “biodegradable” as used herein means degradable under expected conditions of in vivo use (e.g. insertion into skin), irrespective of the mechanism of biodegradation. Exemplary mechanisms of biodegradation include disintegration, dispersion, dissolution, erosion, hydrolysis, and enzymatic degradation.

Examples of microprojections comprising antigens are disclosed in WO2008/130587 and WO2009/048607. Methods of manufacture of metabolisable microneedles are disclosed in WO2008/130587 and WO2010/124255. Coating of microprojections with antigen can be performed by any method known to the skilled person for example by the methods disclosed in WO06/055844, WO06/055799.

Suitable delivery devices for cutaneous delivery including intradermal delivery, in the methods and uses described herein include the BD Soluvia™ device which is a microneedle device for intradermal administration, the Corium MicroCor™ patch delivery system, the Georgia Tech microneedle vaccine patch, the Nanopass microneedle delivery device and the Debiotech Nanoject™ microneedle device. Also provided is a cutaneous or intradermal delivery device containing an immunogenic composition for RSV maternal immunization as described herein, for example a recombinant F protein analog such as PreF antigen, optionally formulated with an adjuvant such as a mineral salt e.g. alum, or QS21, or 3D-MPL or a combination thereof.

### EXAMPLES

### Example 1

### Proof of Concept—Maternal Immunization in a Guinea Pig Model

The guinea pig model was selected as placental structure and IgG transfer is closer to that of humans than is the case for typical rodent models (reviewed in Pentsuk and van der Laan (2009) Birth Defects Research (part B) 86:328-344. The relatively long gestational period of the guinea pig (68 days) allows for immunization and immune response development during pregnancy. In order to mimic the RSV immune status of pregnant women who have been in exposed to RSV throughout their lives and have a pre-existing immune response to RSV, female guinea pigs were primed with live RSV at either 6 weeks or 10 weeks prior to vaccination.

Female guinea pigs (N=5/group) were primed intranasally with live RSV virus (2.5×10⁵ pfu), 6 or 10 weeks prior to vaccination (approximately at the time of mating or 4 weeks prior to mating). Two groups were left unprimed. Pregnant females were immunized approximately 6 weeks after the start of gestation with 10 μg of PreF antigen combined with aluminum hydroxide. One unprimed group of females was injected with PBS. Serum samples were collected throughout the priming and gestation period to monitor levels of anti-RSV binding and neutralizing antibodies.

Offspring (7- to 16-day old) were challenged intranasally with live RSV at 1×10⁷ pfu. Four days after challenge, lungs were collected and separated into 7 lobes. Virus was titrated in 6 of the 7 lobes and total virus particles per gram of lung were calculated.

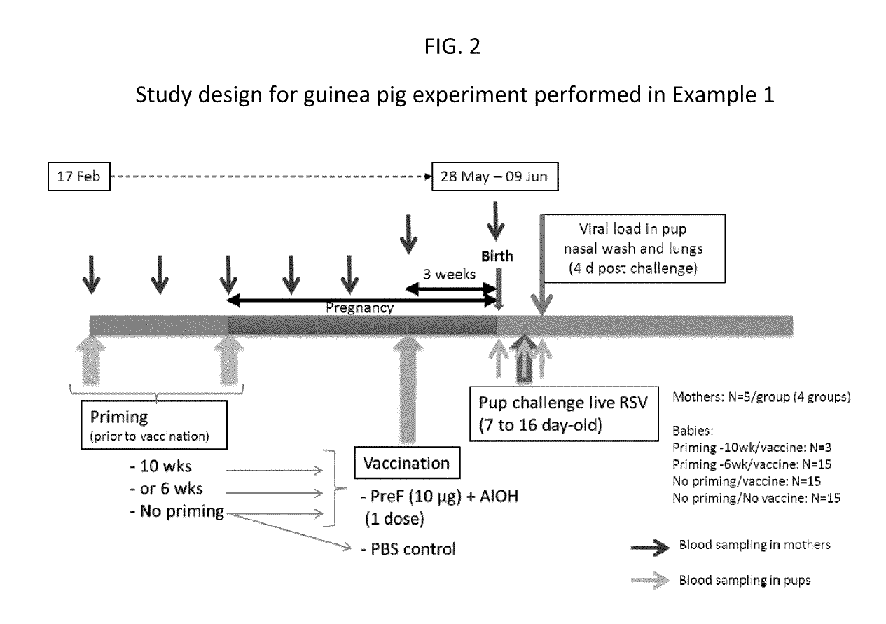

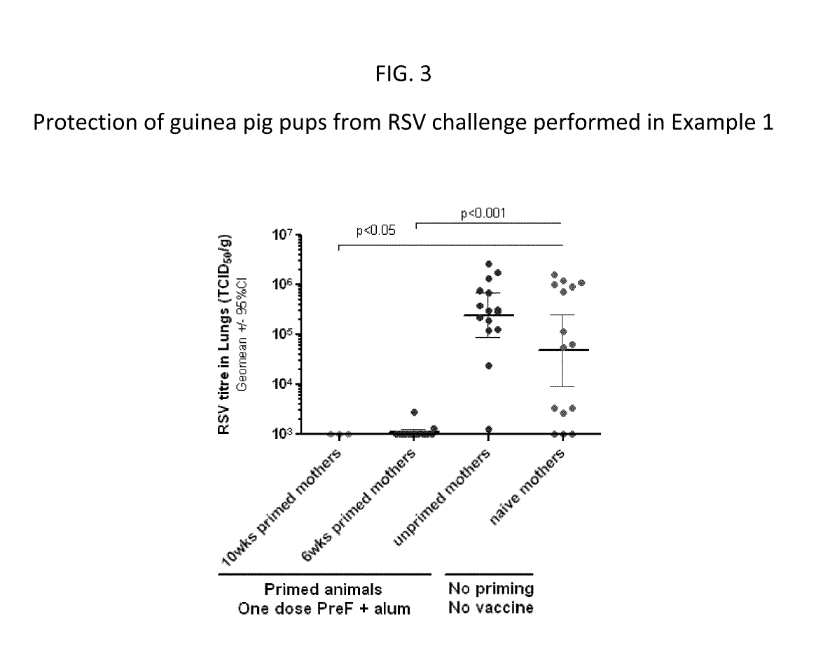

Results are shown in the graph in FIGS. 2 and 3.

Similar levels of antibodies were observed on the day of vaccination whether guinea pigs had been primed 6 or 10 weeks earlier. Plateau titres were reached as of 14 days post priming. Neutralising antibody titres do not decline after reaching a plateau for at least about 60 days. Thus priming at both time points was equivalent in this model and suitable to mimic maternal infection in humans.

Results from lung viral load in the guinea pig offspring indicate that offspring born to primed and vaccinated mothers were protected from RSV challenge, as compared to offspring born to unprimed/unvaccinated mothers. In contrast, offspring born to unprimated/vaccinated mothers were not protected from RSV challenge.

We claim:

1. A method for protecting an infant against infection or disease caused by respiratory syncytial virus (RSV), the method comprising:
administering to a pregnant female with a gestational infant an immunogenic composition capable of boosting an RSV-specific humoral immune response, which immunogenic composition comprises a recombinant RSV antigen comprising an F protein analog,
wherein at least one subset of RSV-specific antibodies elicited or increased in the pregnant female by the immunogenic composition are transferred via the placenta to the gestational infant, thereby protecting the infant against infection or disease caused by RSV.

2. The method of claim 1, wherein the at least one subset of RSV-specific antibodies is detectable at a level at or greater than 30 mcg/mL in the infant's serum at birth.

3. The method of claim 1, wherein the at least one subset of RSV-specific antibodies transferred via the placenta are neutralizing antibodies.

4. The method of claim 1, wherein the at least one subset of antibodies comprises IgG antibodies.

5. The method of claim 4, wherein the at least one subset of RSV-specific antibodies transferred via the placenta comprises IgG₁ antibodies.

6. The method of claim 5, wherein the at least one subset of RSV-specific antibodies transferred via the placenta comprises IgG₁ neutralizing antibodies.

7. The method of claim 1, further comprising administering to the infant at least one composition that primes or induces an active immune response against RSV in the infant.

8. The method of claim 7, wherein the at least one composition administered to the infant comprises an RSV antigen comprising an F protein analog.

9. The method of claim 7, wherein the at least one composition administered to the infant comprises a nucleic acid, a recombinant viral vector or a viral replicon particle, which nucleic acid, recombinant viral vector or viral replicon particle encodes at least one RSV protein antigen or antigen analog.

10. The method of claim 7, wherein the infant is immunologically immature.

11. The method of claim 1, wherein the F protein analog is a PreF antigen that includes at least one modification that stabilizes the prefusion conformation of the F protein.

12. The method claim 1, wherein the infant is less than six months of age.

13. The method of claim 1, wherein protecting the infant comprises reducing the incidence or severity of infection or disease caused by RSV.

14. The method of claim 1, wherein the infection or disease caused by RSV comprises lower respiratory tract infection (LRTI) which is reduced by incidence or severity.

15. The method of claim 1, wherein the pregnant female is at 26 weeks of gestation or later.

16. The method of claim 1, wherein the F protein analog comprises in an N-terminal to C-terminal direction: an F₂ domain and an F₁ domain of an RSV F protein polypeptide, and a heterologous trimerization domain, wherein there is no furin cleavage site between the F₂ domain and the F₁ domain.

17. The method of claim 16, wherein the F protein analog comprises at least one modification selected from:
(i) a modification that alters glycosylation.
(ii) a modification that eliminates at least one non-furin cleavage site;
(iii) a modification that deletes one or more amino acids of the pep27 domain; and
(iv) a modification that substitutes or adds a hydrophilic amino acid in a hydrophobic domain of the F protein extracellular domain.

18. The method of claim 16, wherein the F₂ domain comprises an RSV F protein polypeptide corresponding to amino acids 26-105 and/or wherein the F₁ domain comprises an RSV F protein polypeptide corresponding to amino acids 137-516 of the reference F protein precursor polypeptide (F₀) of SEQ ID NO:2.

19. The method of claim 1, wherein the F protein analog is selected from the group of:
a) a polypeptide comprising a polypeptide selected from the group of SEQ ID NO:6, SEQ ID NO:8, SEQ ID NO:10, SEQ ID NO:18, SEQ ID NO:20 and SEQ ID NO:22;
b) a polypeptide encoded by a polynucleotide selected from the group of SEQ ID NO:5, SEQ ID NO:7, SEQ ID NO:9, SEQ ID NO:17, SEQ ID NO:19 and SEQ ID NO:21, or by a polynucleotide sequence that hybridizes under stringent conditions over substantially its entire length to a polynucleotide selected from the group of SEQ ID NO:5, SEQ ID NO:7, SEQ ID NO:9, SEQ ID NO:17, SEQ ID NO:19 and SEQ ID NO:21, which polypeptide comprises an amino acid sequence corresponding at least in part to a naturally occurring RSV strain;
c) a polypeptide with at least 95% sequence identity to a polypeptide selected from the group of SEQ ID NO:6, SEQ ID NO:8, SEQ ID NO:10, SEQ ID NO:18, SEQ ID NO:20 and SEQ ID NO:22, which polypeptide comprises an amino acid sequence that does not correspond to a naturally occurring RSV strain.

20. The method of claim 1, wherein the F protein analog is formulated in an immunogenic composition comprising an adjuvant.

21. The method of claim 20, wherein the adjuvant comprises at least one of: 3D-MPL, QS21, an oil-in-water emulsion, and a mineral salt.

22. The method of claim 21, wherein the adjuvant comprises QS21.

23. The method of claim 21, wherein the adjuvant comprises alum.

24. The method of claim 1, wherein the immunogenic composition is formulated without an adjuvant.

25. The method of claim 1, wherein the immunogenic composition is administered by an intramuscular, cutaneous or intradermal administration route.

26. The method of claim 1, wherein the immunogenic composition comprising the F protein analog is administered to the pregnant female with at least one additional antigen or immunogenic composition selected from the group of: influenza, hMPV, PIV, pertussis, diphtheria and tetanus.
